(19) United States
(12) Patent Application Publication
(10) Pub. No.: US 2024/0117042 A1
(43) Pub. Date: Apr. 11, 2024

(54) IMMUNOHISTOCHEMISTRY METHODS AND KIR3DL2-SPECIFIC REAGENTS

(71) Applicant: INNATE PHARMA, MARSEILLE (FR)

(72) Inventors: BENJAMIN ROSSI, MARSEILLE (FR); STÉPHANIE CHANTEUX, MARSEILLE (FR); ROMAIN REMARK, MARSEILLE (FR); CÉCILE BONNAFOUS, MARSEILLE (FR); CLARENCE DEFFAUD, VOIRON (FR); THIBAUT PELAT, IZEAUX (FR)

(21) Appl. No.: 18/285,668

(22) PCT Filed: Apr. 4, 2022
(86) PCT No.: PCT/EP2022/058885

§ 371 Date: Oct. 5, 2023

Related U.S. Application Data
(60) Provisional application No. 63/170,603, filed on Apr. 5, 2021.

(51) Int. Cl. C07K 16/28

(52) U.S. Cl. CPC C07K 16/2803; C07K 2317/622

(57) ABSTRACT

The present invention relates to antibodies and methods for detecting KIR3DL2 expression in paraffin-embedded tissue samples. Also provide are methods of making antibodies, antibody fragments, and derivatives thereof that specifically bind to their target antigen in paraffin-embedded tissue samples.

### CROSS-REFERENCE TO RELATED APPLICATIONS

This application claims the benefit of U.S. Provisional Application No. 63/170,603 filed 5 Apr. 2021; which is incorporated herein by reference in its entirety; including any drawings.

### REFERENCE TO SEQUENCE LISTING

The present application is being filed along with a Sequence Listing in electronic format. The Sequence Listing is provided as a file entitled “KIR-12 PCT Sequences_ST25”, created 30 Mar. 2022, which is 25.7 KB in size. The information in the electronic format of the Sequence Listing is incorporated herein by reference in its entirety.

### FIELD OF THE INVENTION

The present invention relates to research and diagnostic tools to detect KIR3DL2 in biological sample (e.g. paraffin embedded tissue samples). The invention also relates to methods of using said tools to detect KIR3DL2-expressing cells.

### BACKGROUND

Killer immunoglobulin-like receptors (KIR) are a family of receptors that, along with C-type lectin receptors (CD94-NKG2), used by human NK cells and T-lymphocyte subsets to specifically recognize MHC class I molecules.

Among the member of the killer-cell immunoglobulin (Ig)-like receptor (KIR) family, Killer-cell immunoglobulin-like receptor, 3 Ig domains and long cytoplasmic tail 2 (KIR3DL2) has been studied as a target for the treatment of malignancies involving CD4+ T cells that express KIR3DL2 receptors, particularly CD4+ T cells, including cutaneous T cell lymphomas (CTCL) such as Mycosis Fungoides and Sezary Syndrome (see, e.g. WO2010/081890 and WO02/50122). KIR3DL2 receptor is also often expressed by tumor cells in other peripheral T-cell lymphoma (PTCL) and at low frequency on normal lymphocytes.

Monoclonal antibodies targeting KIR3DL2 receptors and presenting an increased activity in the treatment of KIR3DL2-expressing cells malignancies, particularly CD4+ T cells malignancies has been developed (see e.g. WO2014/044686).

In order to better understand the tumor environment it is often desirable to detect KIR3DL2 receptors present in tumor tissue. This can be done for example using frozen tissue samples. This is not only useful in research but can also help in the decision about what type of treatment to use, for example, by detecting whether a tissue (e.g. a tumor environment) is characterized by the presence of a protein that is the target of a treatment (e.g. an immunotherapy). The information can be valuable in order to select a treatment that is capable of modulating the activity of the protein and/or of the cells expressing it.

Some antibodies suitable for the detection of KIR3DL2 polypeptides in cell culture or in frozen tissue samples are already known. Antibody 12B111 and 19H12 are indeed suitable for use in detection (e.g. in vitro assays) of KIR3DL2 expression on the surface of tumor cells because 12B111 and 19H12 are both able to detect KIR3DL2-positive cells in detection assays. 12B111 is advantageous for immunohistochemistry assays using frozen tissue sections, while 19H12 is advantageous for flow cytometry detection.

The access to frozen tissue samples being a limiting factor, there is a need of developing new test for detecting KIR3DL2 in other samples such as tissue samples that have been preserved as formalin-fixed paraffin embedded (FFPE) samples. Unfortunately, it had often been impossible to find KIR3DL2 specific monoclonal antibodies that work effectively and with specificity in FFPE sections. This is believed to be due to the impact of the formalin fixation on structure of proteins. Epitopes bound by antibodies described as being specific on recombinant protein or cells are often present on other proteins when used in FFPE, rendering the antibodies non-specific. In other cases, many epitopes on native cellular protein are destroyed by formaldehyde (e.g. formalin) fixation, causing antibodies identified using recombinant protein or cells to be ineffective for staining FFPE sections.

There is therefore a need for improved antibodies targeting specifically KIR3DL2 for use in staining biological sample (e.g. paraffin embedded tissue sections).

### SUMMARY OF THE INVENTION

The invention relates, inter alia, to the study, detection and/or monitoring of KIR3DL2 polypeptides in biological sample, preferably formalin-fixed paraffin embedded (FFPE) tissue samples. The present disclosure arises from the characterization of antibodies suitable for detecting KIR3DL2 in biological sample. The antibodies retain specificity for such predetermined target antigen in FFPE protocols, notably they bind an epitope on KIR3DL2 polypeptide that remain present and specific following formalin fixation. The antibodies permitted high specificity of detection of KIR3DL2 in IHC protocols, without detecting KIR3DL1 and/or other KIR polypeptides (e.g., KIR3DS1). The resulting diagnostic antibodies can serve as a reagent for consistent detection of in FFPE samples from individuals.

The KIR3DL2-specific antibodies described herein were found through the design and testing of synthetic peptides designed to mimic potential formalin-modified epitopes that could arise in KIR3DL2 proteins. Antibodies that bound to certain synthetic peptides were in turn found to be selective for KIR3DL2 over KIR3DL1 in FFPE samples. The study thereby identified new epitopes or binding sites that are present or arise in formalin treated KIR3DL2, as well as their corresponding sites or sequence in the wild-type KIR3DL2 amino acid sequence.

Formalin-fixed paraffin-embedded (FFPE) tissue provides two main advantages over other immunologic methods: (1) the tissue does not require special handling; and (2) cytologic and architectural features are well perceived, allowing for improved histopathologic interpretation. However, it was often impossible to find KIR3DL2 specific monoclonal antibodies that work effectively and with specificity in FFPE sections. Antibodies previously developed were indeed not KIR3DL2 specific as they bound also to KIR3DL1 polypeptide when tested in FFPE samples. The inventors have developed antibodies suitable for detecting specifically KIR3DL2 polypeptide and for use in staining of FFPE biological samples. The resulting antibodies were tested in a variety of KIR3DL2-expressing cells (e.g. transfected cells, human tissues from human donors or tumor tissues from patients) and found to retain excellent performance in detection of target antigen in FFPE tissue section.

The present disclosure therefore provides an antibody or antibody fragment thereof capable of binding to a KIR3DL2 polypeptide in a biological sample, wherein the antibody or antibody fragment comprises a heavy chain comprising an amino acid sequence at least 80%, 85%, 90%, 95%, 96%, 97%, 98% or 99% identical to the amino acid sequence of SEQ ID NO: 21, and a light chain comprising an amino acid sequence at least 80% identical to the amino acid sequence of SEQ ID NO: 22.

Another object of the present disclosure is an antibody or antibody fragment thereof capable of binding to a KIR3DL2 polypeptide, wherein the antibody or antibody fragment comprises the heavy chain CDR1, 2 and 3 of the heavy chain variable region shown in SEQ ID NO: 21, and the light chain CDR1, 2 and 3 of the light chain variable region shown in SEQ ID NO: 22.

In one embodiment, provided is an antibody or antibody fragment comprising: (i) a heavy chain comprising CDR 1, 2 and 3 (HCDR1, HCDR2, HCDR3) having a sequence of SEQ ID NO: 3 (HCDR1), SEQ ID NO: 6 (HCDR2) and SEQ ID NO: 9 (HCDR3), and (ii) a light chain comprising CDR 1, 2 and 3 (LCDR1, LDR2, LCDR3) having a sequence of SEQ ID NO: 12 (LCDR1), SEQ ID NO: 15 (LCDR2), and SEQ ID NO: 18 (LCDR3), wherein each CDR may optionally comprise 1, 2 or 3 amino acid substitutions, deletions or insertions.

In one embodiment, provided is a monoclonal antibody or antibody fragment that is a function-conservative variant of antibody P3-R4D-H5. In one embodiment, provided is an antibody or antibody fragment that is a function-conservative variant of an antibody having the heavy chain variable region of SEQ ID NO: 21, and the light chain CDR1, 2 and 3 of the light chain variable region of SEQ ID NO: 22.

In one embodiment, provided an isolated polypeptide (e.g. an isolated polypeptide comprises or corresponding to an epitope on KIR3DL2 present following formalin treatment of KIR3DL2-expressing cells), wherein the isolated polypeptide consists of the amino acid sequence of SEQ ID NO:24. In another embodiment, the isolated polypeptide according to the disclosure can be modified or fused to one or more heterologous polypeptides or comprised in another polypeptide (e.g. a polypeptide comprising one or more non-KIR3DL2 amino acid sequences.

In one embodiment, provided is an antibody or antibody fragment thereof that binds such an isolated polypeptide. In one embodiment, binding of said antibody or antibody fragment thereof to said isolated polypeptide is determined by an ELISA test in which said antibody or antibody fragment thereof is contacted with a polypeptide comprising the amino acid sequence of SEQ ID NO: 24. In a particular embodiment, said polypeptide of amino acid sequence of SEQ ID NO: 24 is bound on a solid support, e.g. such polypeptide can thus be fused to a linker peptide bound to a solid support.

In one embodiment, provided is a monoclonal antibody or antibody fragment that binds to a polypeptide comprising or consisting of an amino acid sequence selected from the group consisting of CEHFFLHREGISEDPSRLVG (SEQ ID NO: 23), CTPLTDTSVYTELPNAEPRS (SEQ ID NO: 24) and CPRAPQSGLEGVF (SEQ ID NO: 25). In one embodiment, binding of said antibody or antibody fragment thereof to said polypeptide is determined by an ELISA test in which said antibody or antibody fragment thereof is contacted with said polypeptide comprising the amino acid sequence of SEQ ID NO: 23, or SEQ ID NO: 24, or SEQ ID NO: 25. In a particular embodiment, said polypeptide of amino acid sequence of SEQ ID NO: 23, SEQ ID NO: 24, or SEQ ID NO: 25 is bound on a solid support.

In one embodiment, provided is a monoclonal antibody or antibody fragment that binds a KIR3DL2 polypeptide, wherein the antibody or antibody fragment binds to a polypeptide comprising or consisting of an amino acid sequence selected from the group consisting of CEHFFLHREGISEDPSRLVG (SEQ ID NO: 23), CTPLTDTSVYTELPNAEPRS (SEQ ID NO: 24) and CPRAPQSGLEGVF (SEQ ID NO: 25).

In one embodiment, provided is a monoclonal antibody or antibody fragment that binds the same epitope on KIR3DL2 as an antibody having the heavy chain variable region of SEQ ID NO: 21, and the light chain CDR1, 2 and 3 of the light chain variable region of SEQ ID NO: 22. Optionally, the antibody or antibody fragment binds a polypeptide comprising or consisting of an amino acid sequence selected from the group consisting of CEHFFLHREGISEDPSRLVG (SEQ ID NO: 23), CTPLTDTSVYTELPNAEPRS (SEQ ID NO: 24) and CPRAPQSGLEGVF (SEQ ID NO: 25). In any embodiment herein, the antibody or antibody fragment thereof according to the disclosure is capable of specifically binding to a KIR3DL2 polypeptide in a formalin-fixed paraffin-embedded (FFPE) tissue sample.

In another embodiment provided is an antibody or antibody fragment that binds to an intracellular portion or epitope of a KIR3DL2 polypeptide in a biological sample. For example antibody or antibody fragment can be specified as binding to the cytoplasmic domain of a KIR3DL2 polypeptide (or to an epitope in such domain), optionally wherein the cytoplasmic domain corresponds to residues 340-434 of SEQ ID NO: 1. In one embodiment, the antibody or antibody fragment binds to an portion or epitope of a KIR3DL2 polypeptide that corresponds to residues 399-417 of SEQ ID NO: 1 In one embodiment the antibody or antibody fragment binds to an intracellular portion or epitope of a KIR3DL2 polypeptide in a biological sample having been fixed in formalin, then cut into sections before being into contact with said antibody or antibody fragment. In one embodiment, said intracellular portion or epitope of KIR3DL2 polypeptide has the amino acid sequence TPLTDTSVYTELPNAEPRS (SEQ ID NO: 31). In one embodiment, the antibody or antibody fragment binds to a polypeptide comprising or consisting of the amino acid sequence CTPLTDTSVYTELPNAEPRS (SEQ ID NO: 24).

In a further embodiment provided is an antibody or antibody fragment that binds to a KIR3DL2 polypeptide in a biological sample, and said antibody or antibody fragment thereof does not substantially bind to a KIR3DL1 polypeptide (e.g. KIR3DL1 allele *00101 comprising the amino acid sequence shown in SEQ ID NO: 30. Preferably, said biological sample is a formalin-fixed paraffin-embedded tissue sample.

In any embodiment herein, the antibody or antibody fragment can be specified as being capable of specifically binding to a KIR3DL2 polypeptide in a biological sample of cells that have been prepared as a paraffin-embedded cell pellet.

In one embodiment, the antibody or antibody fragment is capable of binding (or, e.g., staining) KIR3DL2-expressing cells that have been prepared as a paraffin-embedded cell pellet, wherein said antibody or antibody fragment thereof does not bind (or, e.g. stain) KIR3DL1-expressing cells that do not express KIR3DL2 and that have been prepared as a paraffin-embedded cell pellet, optionally further wherein the cells are fixed in formalin, then cut into sections before being brought into contact with said antibody or antibody fragment thereof.

The present disclosure also provides antibody or antibody fragment thereof for use in detecting the presence of a KIR3DL2-expressing cell in a biological sample. In one embodiment, the biological sample is a tissue sample. In one embodiment, the biological sample is a fixed tissue sample. In a further one embodiment, the biological sample is a formalin-fixed paraffin-embedded (FFPE) tissue sample. In one embodiment, the detection of the presence of a KIR3DL2 expressing cell by the antibody or antibody fragment thereof according to the disclosure is done by means of immunohistochemistry (IHC).

Advantageously, the antibody or antibody fragment thereof for use in detecting KIR3DL2-expressing cells by immunostaining of paraffin-embedded tissue sections, said antibody or antibody fragment thereof remaining specific in FFPE and with advantageous affinity, permitting accurate detection of KIR3DL2.

In another aspect provided is an in vitro method of detecting a KIR3DL2-expressing cell in a sample, said method comprising (i) providing a biological sample from an individual comprising cells, and (ii) detecting KIR3DL2-expressing cells with the antibody or the antibody fragment thereof according to the disclosure. In one embodiment, the biological sample is a tissue sample. In one embodiment, the biological sample is a fixed tissue sample. In a further one embodiment, the biological sample is a formalin-fixed paraffin-embedded (FFPE) tissue sample. In one embodiment, the step (ii) of detecting KIR3DL2-expressing cells with the antibody or the antibody fragment thereof according to the disclosure comprises contacting the sample with the antibody or the antibody fragment as disclosed and detecting the formation of immunological complexes resulting from the immunological reaction between said antibody or antibody fragment thereof and the sample. In one embodiment, the detection of the formation of such immunological complexes between the antibody or antibody fragment thereof according to the disclosure and the sample is done by means of immunohistochemistry (IHC). In one embodiment, the detection of the formation of such immunological complexes between the antibody or antibody fragment thereof according to the disclosure and the sample is done by using a secondary antibody that specifically binds to the antibody or antibody fragment of the disclosure. In one embodiment, the paraffin-embedded tissue sample has been fixed, embedded in paraffin, sectioned, deparaffinized, and transferred to a slide.

Another aspect of the disclosure is an in vitro method of assessing the suitability of an individual having a cancer for treatment with an immunotherapeutic agent, said method comprising (i) providing a biological sample from a patient, and (ii) detecting KIR3DL2-expressing cells in said sample using an antibody or antibody fragment according to the disclosure, wherein a detection of KIR3DL2-expressing cells indicates that the individual is suitable for treatment with an immunotherapeutic agent. In one embodiment, the biological sample is a tissue sample. In one embodiment, the biological sample is a fixed tissue sample. In a further one embodiment, the biological sample is a formalin-fixed paraffin-embedded (FFPE) tissue sample. In one embodiment, the immunotherapeutic agent for treating an individual having a cancer is an agent that binds a KIR3DL2 polypeptide. In one embodiment, the immunotherapeutic agent that binds a KIR3DL2 polypeptide is an antibody that binds a KIR3DL2 polypeptide and enhances cytotoxicity through ADCC against KIR3DL2 expressing cells. In a preferred embodiment, the antibody is LACUTAMAB. In a further embodiment, the paraffin-embedded tissue sample has been fixed, embedded in paraffin, sectioned, deparaffinized, and transferred to a slide.

The disclosure also provides a method of treating a disease in an individual, said method comprising: (i) providing a biological sample from an individual, (ii) detecting a KIR3DL2-expressing cells in said sample using an antibody or antibody fragment thereof according to the disclosure, and (iii) if KIR3DL2-expressing cells are detected, administering to the individual an immunotherapeutic agent. In one embodiment, the biological sample is a tissue sample. In one embodiment, the biological sample is a fixed tissue sample. In a further one embodiment, the biological sample is a formalin-fixed paraffin-embedded (FFPE) tissue sample. In one embodiment, the disease to be treated is a cancer. In one embodiment, the cancer is a lymphoma, e.g. a CD4+ T cell lymphoma. In one embodiment, the CD4+ lymphoma is a cutaneous T cell lymphoma (CTCL). In one embodiment, the CTCL is mycosis fungoides or Sezary syndrome. In an additional embodiment, the CTCL is a transformed T lymphoma. In another embodiment, the CD4+ lymphoma is a peripheral T cell lymphoma (PTCL). In one embodiment, the paraffin-embedded biological sample has bee, fixed, embedded in paraffin, sectioned, deparaffinized, and transferred to a slide.

Another aspect of the disclosure concerns a kit comprising the antibody or antibody fragment according to the disclosure, and a labelled secondary antibody that specifically recognizes said antibody or antibody fragment thereof according to the disclosure.

The disclosure also provides an isolated nucleic acid encoding the antibody or antibody fragment according to the disclosure.

Also provided is a hybridoma or a recombinant host cell producing the antibody or antibody fragment according to the disclosure.

These and additional advantageous aspects and features of the invention may be further described elsewhere herein.

### BRIEF DESCRIPTION OF THE DRAWING

FIG. 1 represents the optical density (450 nm) measured in Elisa assay on several dilutions of clone P3-R4D-H5 soluble scFv selected from scFv library and against the peptide 3 coupled with BSA or free, or against BSA.

FIG. 2 represents pictures of IHC staining obtained with anti-KIR3DL2 soluble scFv clones P3-R4D-H5, P3-R4D-F4, P3-R4D-C10, P3-R4D-C1, P3-R4D-B9, P3-R4D-B5 on KIR3DL2- and KIR3DL1-expressing cell pellets. Staining was performed at 5 μg/mL on a Leica Bond RX.

FIG. 3 represents pictures of IHC staining obtained with anti-KIR3DL2 clone P3-R4D-H5 or an isotype control antibodies on different CHO/CHO-mb-HuKIR3DL2 mixed FFPE cell pellets. Staining was performed at 5 μg/mL on a Leica Bond RX. Scale bars represent 25 μm. CHO are not expressing KIR3DL2, whereas CHO-mb-HuKIR3DL2 are KIR3DL2-expressing cells. As stated on the figure Ab means antibody; FFPE means formalin-fixed paraffin-embedded; IC means isotype control and IHC means immunohistochemistry.

FIG. 4 represents pictures of IHC staining obtained with anti-KIR3DL2 clone P3-R4D-H5 or isotype control antibodies on different Raji/HuT mixed FFPE cell pellets. Staining was performed at 5 μg/mL on a Leica Bond RX. Scale bars represent 25 μm. Arrowhead represents KIR3DL2+ cells. As stated on the figure Ab means antibody; FFPE means formalin-fixed paraffin-embedded; IC means isotype control and IHC means immunohistochemistry.

FIGS. 5A, 5B and 5C represent pictures of IHC staining obtained with anti-KIR3DL2 clone 12B11 on KIR3DL2-expressing RAJI cells (and non-expressing KIR3DL2 RAJI cells as negative control). Several staining conditions were tested: Citrate Buffer pH7, kit envision+DAB; Citrate Buffer pH7, kit envision+tyramide-biotine+Streptavidine-HRP+DAB; Citrate Buffer pH6, kit envision+DAB; EDTA pH7 kit envision+DAB; Tris-EDTA pH9 kit envision+DAB.

FIG. 6 is a graph representing the optical density measured on FFPE samples stained by several concentration of anti-KIR3DL2 clone P3-R4D-H5 in IHC. Staining was performed on several FFPE samples: CHO-K1SV cells (CHO), CHO-K1SV-mb-HuKIR3DL1 cells (CHO-KIR3DL1), and CHO-K1SV-mb-HuKIR3DL2 cells (CHO-KIR3DL2).

FIG. 7 represents images of KIR3DL2 chromogenic IHC stainings on different CTCL biopsies from individual suffering of CTCL (e.g. Mycosis Fungoides or Sezary syndrome). FFPE sections were stained with the clone P3-R4D-H5 Ab at 5 μg/mL on a Leica Bond RX (left panel) and frozen sections were stained using the anti-KIR3DL2 clone 12B11 at 10 μg/mL on a Ventana Benchmark XT (right panel). For each cases, low and high magnifications are shown and the percentages of KIR3DL2+ cells among mononuclear cells are indicated. Scale bars correspond to 1 or 2.5 mm on low magnification images (for middle left, lower left and lower right panels or middle right, upper left and upper right panels, respectively) and to 50 μm on high magnification images. As stated on the figure B means biopsy; CTCL means cutaneous T cell lymphoma; FFPE means formalin-fixed paraffin-embedded; and IHC means immunohistochemistry.

FIG. 8 represents images of KIR3DL2 chromogenic IHC stainings on PTCL biopsies from individual suffering of PTCL. FFPE sections were stained with the clone P3-R4D-H5 Ab at 5 μg/mL on a Leica Bond RX (left panel) and frozen sections were stained using the anti-KIR3DL2 clone 12B11 at 10 μg/mL on a Ventana Benchmark XT (right panel). For the 2 cases, low and high magnifications are shown and the percentages of KIR3DL2+ cells among mononuclear cells determined by pathologist are indicated. Scale bars correspond to 2.5 mm on low magnification images and to 50 μm on high magnification images. As stated in the figure PTCL means peripheral T cell lymphoma; FFPE means formalin-fixed paraffin-embedded; IHC means immunohistochemistry; and LN means lymph node.

FIG. 9 represents images of KIR3DL2 chromogenic IHC stainings on FFPE samples from individuals suffering of CTCL (Mycosis Fungoides). FFPE sections were stained with the clone P3-R4D-H5 Ab at 5 μg/mL on a Leica Bond RX. FIGS. 9A and 9C are IHC images of highly positive CTCL with a strong KIR3DL2 signal. FIG. 9B is an IHC image of a weak positive CTCL. FIG. 9D is an IHC image of a recurrent CTCL (Mycosis Fungoides) sample with regions of strong KIR3DL2 positivity and zones that are almost negative.

FIG. 10 represents images of KIR3DL2 chromogenic IHC stainings on FFPE samples from individuals suffering of PTCL. FFPE sections were stained with the clone P3-R4D-H5 Ab at 5 μg/mL on a Leica Bond RX. FIG. 10A is an IHC image of a highly positive PTCL (not otherwise specified). FIG. 10B is an IHC image of a PTCL (not otherwise specified), with scattered positive tumor cells against a backdrop of faint stromal cell positivity. FIGS. 10C and 10D are IHC images of moderate KIR3DL2 positive PTCL (not otherwise specified), with a regional variability within the sample in FIG. 10D.

### DETAILED DESCRIPTION OF THE INVENTION

### Definitions

As used in the specification, “a” or “an” may mean one or more. As used in the claim(s), when used in conjunction with the word “comprising”, the words “a” or “an” may mean one or more than one.

Where “comprising” is used, this can optionally be replaced by “consisting essentially of”, more optionally by “consisting of”.

The term “antibody” herein is used in the broadest sense and specifically includes full-length monoclonal antibodies, polyclonal antibodies, multispecific antibodies (e.g., bispecific antibodies), and antibody fragments and derivatives, so long as they exhibit the desired biological activity. Various techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988).

An “antibody fragment” comprises a portion of a full-length antibody, e.g. antigen-binding or variable regions thereof. Examples of antibody fragments include Fab, Fab′, F(ab)₂, F(ab′)₂, F(ab)₃, Fv (typically the VL and VH domains of a single arm of an antibody), single-chain Fv (scFv), dsFv, Fd fragments (typically the VH and CH1 domain), and dAb (typically a VH domain) fragments; VH, VL, VhH, and V-NAR domains; minibodies, diabodies, triabodies, tetrabodies, and kappa bodies (see, e.g., III et al., Protein Eng 1997; 10: 949-57); camel IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23, 1126-1136; WO2005040219, and published U.S. Patent Applications 20050238646 and 20020161201.

As used herein, the term “antigen binding domain” refers to a domain comprising a three-dimensional structure capable of immunospecifically binding to an epitope. Thus, in one embodiment, said domain can comprise a hypervariable region, optionally a VH and/or VL domain of an antibody chain, optionally at least a VH domain. In another embodiment, the binding domain may comprise one, two or all three complementarity determining region (CDR) of an antibody chain. In another embodiment, the binding domain may comprise a polypeptide domain from a non-immunoglobulin scaffold.

The term “antibody derivative”, as used herein, comprises a full-length antibody or a fragment of an antibody, e.g. comprising at least antigen-binding or variable regions thereof, wherein one or more of the amino acids are chemically modified, e.g., by alkylation, PEGylation, acylation, ester formation or amide formation or the like. This includes, but is not limited to, PEGylated antibodies, cysteine-PEGylated antibodies, and variants thereof.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. The application of either definition to refer to a CDR of an antibody or variants thereof is intended to be within the scope of the terms as defined and used herein. The appropriate amino acid residues which encompass the CDRs as defined by commonly used numbering schemes are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1

| CDR | Kabat | Chotia | AbM |
|---|---|---|---|
| HCDR1 | 31-35 | 26-32 | 26-35 |
| HCDR2 | 50-65 | 52-58 | 50-58 |
| HCDR3 | 95-102 | 95-102 | 95-102 |
| VCDR1 | 24-34 | 26-32 | 24-34 |
| VCDR2 | 60-56 | 50-52 | 50-56 |
| VCDR3 | 89-97 | 91-96 | 89-97 |

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

By “constant region” as defined herein is meant an antibody-derived constant region that is encoded by one of the light or heavy chain immunoglobulin constant region genes. By “constant light chain” or “light chain constant region” as used herein is meant the region of an antibody encoded by the kappa (Ckappa) or lambda (Clambda) light chains. The constant light chain typically comprises a single domain, and as defined herein refers to positions 108-214 of Ckappa, or Clambda, wherein numbering is according to the EU index (Kabat et al., 1991, Sequences of Proteins of Immunological Interest, 5th Ed., United States Public Health Service, National Institutes of Health, Bethesda). By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

By “Fab” or “Fab region” as used herein is meant the polypeptide that comprises the VH, CH1, VL, and CL immunoglobulin domains. Fab may refer to this region in isolation, or this region in the context of a polypeptide, multispecific polypeptide or antibody, or any other embodiments as outlined herein.

By “single-chain Fv” or “scFv” as used herein are meant antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, New York, pp. 269-315 (1994).

By “Fv” or “Fv fragment” or “Fv region” as used herein is meant a polypeptide that comprises the VL and VH domains of a single antibody.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 (CH2) and Cγ3 (CH3) and the hinge between Cγ1 and Cγ2. Although the boundaries of the Fc region may vary, the human IgG heavy chain Fc region is usually defined to comprise residues C226, P230 or A231 to its carboxyl-terminus, wherein the numbering is according to the EU index. Fc may refer to this region in isolation, or this region in the context of an Fc polypeptide, as described below. By “Fc polypeptide” or “Fc-derived polypeptide” as used herein is meant a polypeptide that comprises all or part of an Fc region. Fc polypeptides include but is not limited to antibodies, Fc fusions and Fc fragments.

By “variable region” as used herein is meant the region of an antibody that comprises one or more Ig domains substantially encoded by any of the VL (including Vkappa (VK) and Vlambda) and/or VH genes that make up the light chain (including kappa and lambda) and heavy chain immunoglobulin genetic loci respectively. A light or heavy chain variable region (VL or VH) consists of a “framework” or “FR” region interrupted by three hypervariable regions referred to as “complementarity determining regions” or “CDRs”. The extent of the framework region and CDRs have been precisely defined, for example as in Kabat (see “Sequences of Proteins of Immunological Interest,” E. Kabat et al., U.S. Department of Health and Human Services, (1983)), and as in Chothia. The framework regions of an antibody, that is the combined framework regions of the constituent light and heavy chains, serves to position and align the CDRs, which are primarily responsible for binding to an antigen.

The term “specifically binds to” means that an antibody or polypeptide can bind preferably in a competitive binding assay to the binding partner, e.g. KIR3DL2, as assessed using either recombinant forms of the proteins, epitopes therein, or native proteins present on the surface of isolated target cells. Competitive binding assays and other methods for determining specific binding are further described below and are well known in the art.

The term “affinity”, as used herein, means the strength of the binding of an antibody or polypeptide to an epitope. The affinity of an antibody is given by the dissociation constant K_{D}, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant K_{A} is defined by 1/K_{D}. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of surface plasmon resonance (SPR) screening (such as by analysis with a BIAcore™ SPR analytical device).

Within the context of this invention a “determinant” designates a site of interaction or binding on a polypeptide.

The term “epitope” refers to an antigenic determinant, and is the area or region on an antigen to which an antibody or polypeptide binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

By “amino acid modification” herein is meant an amino acid substitution, insertion, and/or deletion in a polypeptide sequence. An example of amino acid modification herein is a substitution. By “amino acid modification” herein is meant an amino acid substitution, insertion, and/or deletion in a polypeptide sequence. By “amino acid substitution” or “substitution” herein is meant the replacement of an amino acid at a given position in a protein sequence with another amino acid. For example, the substitution Y50W refers to a variant of a parent polypeptide, in which the tyrosine at position 50 is replaced with tryptophan. A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

An “isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of a polypeptide will exhibit 98%, 98%, or 99% homogeneity for polypeptides in the context of all present peptide species in the composition or at least with respect to substantially active peptide species in the context of proposed use.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

As used herein, “paraffin-embedded sample” (or paraffin-embedded “cells”, “cell pellet”, “slides”, or “tissues”) refers to cells or tissues taken from an organism or from in vitro cell culture that have been fixed, embedded in paraffin, sectioned, deparaffinized, and transferred to a slide. It will be appreciated that fixation and paraffin embedding is a common practice that can vary in many aspects, e.g., with respect to the fixation and embedding methods used, with respect to the protocol followed, etc., and that for the purposes of the present invention any such variant method is encompassed, so long as it involves fixation of the tissue (such as by formalin treatment), embedding in paraffin or equivalent material, sectioning and transfer to a slide.

The term “biological sample” or “sample” as used herein includes but is not limited to a biological fluid (for example serum, lymph, blood), cell sample, or tissue sample (for example bone marrow or tissue biopsy including mucosal tissue such as from the gut, gut lamina propria, or lungs).

In the context herein, “treatment” or “treating” refers to preventing, alleviating, managing, curing or reducing one or more symptoms or clinically relevant manifestations of a disease or disorder, unless contradicted by context. For example, “treatment” of an individual in whom no symptoms or clinically relevant manifestations of a disease or disorder have been identified is preventive or prophylactic therapy, whereas “treatment” of an individual in whom symptoms or clinically relevant manifestations of a disease or disorder have been identified generally does not constitute preventive or prophylactic therapy.

The term “KIR3DL2” (CD158k) refers to a disulphide-linked homodimer of three-Ig domain molecules of about 140 kD, described in Pende et al. (1996) J. Exp. Med. 184: 505-518, the disclosure of which is incorporated herein by reference. Several allelic variants have been reported for KIR3DL2 polypeptides, each of these are encompassed by the term KIR3DL2. The amino acid sequence of the mature human KIR3DL2 (allele *002) is shown in SEQ ID NO: 1, below, corresponding to Genbank accession no. AAB52520 in which the 21 amino acid residue leader sequence has been omitted.

TABLE 2

| | |
|---|---|
| SEQ | LMGGQDKPF LSARPSTVVP RGGHVALQCH YRRGFNNFML YKEDRSHVPI |
| ID NO: | FHGRIFQESF IMGPVTPAHA GTYRCRGSRP HSLTGWSAPS NPLVIMVTGN |
| 1 | HRKPSLLAHP GPLLKSGETV ILQCWSDVMF EHFFLHRDGI SEDPSRLVGQ |
| | IHDGVSKANF SIGPLMPVLA GTYRCYGSVP HSPYQLSAPS DPLDIVITGL |
| | YEKPSLSAQP GPTVQAGENV TLSCSSWSSY DIYHLSREGE AHERRLRAVP |
| | KVNRTFQADF PLGPATHGGT YRCFGSFRAL PCVWSNSSDP LLVSVTGNPS |
| | SSWPSPTEPS SKSGICRHLH VLIGTSVVIF LFILLLFFLL YRWCSNKKNA |
| | AVMDQEPAGD RTVNRQDSDE QDPQEVTYAQ LDHCVFIQRK ISRPSQRPKT |
| | PLTDTSVYTE LPNAEPRSKV VSCPRAPQSG LEGVF |

The cDNA of KIR3DL2 (allele *002) is shown in Genbank accession no. U30272. The amino acid sequence of a human KIR3DL2 allele *003 is shown below, corresponding to Genbank accession no. AAB36593:

TABLE 3

| | |
|---|---|
| SEQ | MSLTVVSMAC VGFFLLQGAW PLMGGQDKPF LSARPSTVVP RGGHVALQCH |
| ID NO: | YRRGFNNFML YKEDRSHVPI FHGRIFQESF IMGPVTPAHA GTYRCRGSRP |
| 2 | HSLTGWSAPS NPVVIMVTGN HRKPSLLAHP GPLLKSGETV ILQCWSDVMF |
| | EHFFLHREGI SEDPSRLVGQ IHDGVSKANF SIGPLMPVLA GTYRCYGSVP |
| | HSPYQLSAPS DPLDIVITGL YEKPSLSAQP GPTVQAGENV TLSCSSWSSY |
| | DIYHLSREGE AHERRLRAVP KVNRTFQADF PLGPATHGGT YRCFGSFRAL |
| | PCVWSNSSDP LLVSVTGNPS SSWPSPTEPS SKSGICRHLH VLIGTSVVIF |
| | LFILLLFFLL YRWCSNKKNA AVMDQEPAGD RTVNRQDSDE QDPQEVTYAQ |
| | LDHCVFIQRK ISRPSQRPKT PLTDTSVYTE LPNAEPRSKV VSCPRAPQSG |
| | LEGVF |

Also encompassed are any nucleic acid or protein sequences sharing one or more biological properties or functions with wild type, full length KIR3DL2 respectively, and sharing at least 70%, 80%, 90%, 95%, 96%, 97%, 98%, 99%, or higher nucleotide or amino acid identity.

As used herein the term KIR3DL1 refers to another KIR3D receptor, that share relatively high amino acid identity with KIR3DL2 and various HLA ligands that bind KIR3DL2 are also recognized by KIR3DL1. Such KIR3DL1 polypeptides can be KIR3DL1 allele *00101, that comprises the amino acid sequence shown in SEQ ID NO: 30.

Whenever within this whole specification “treatment” or “treatment of cancer” or the like is mentioned with reference to an anti-KIR3DL2 binding agent (e.g. antibody), there is meant: (a) method of treatment of cancer, said method comprising the step of administering (for at least one treatment) an anti-KIR3DL2 binding agent, (preferably in a pharmaceutically acceptable carrier material) to an individual, a mammal, especially a human, in need of such treatment, in a dose that allows for the treatment of cancer, (a therapeutically effective amount), preferably in a dose (amount) as specified herein; (b) the use of an anti-KIR3DL2 binding agent for the treatment of cancer, or an anti-KIR3DL2 binding agent, for use in said treatment (especially in a human); (c) the use of an anti-KIR3DL2 binding agent for the manufacture of a pharmaceutical preparation for the treatment of cancer, a method of using an anti-KIR3DL2 binding agent for the manufacture of a pharmaceutical preparation for the treatment of cancer, comprising admixing an anti-KIR3DL2 binding agent with a pharmaceutically acceptable carrier, or a pharmaceutical preparation comprising an effective dose of an anti-KIR3DL2 binding agent that is appropriate for the treatment of cancer; or (d) any combination of a), b), and c), in accordance with the subject matter allowable for patenting in a country where this application is filed.

Examples of antibodies that bind human KIR3DL2 include, but are not limited to antibody AZ158, antibody 19H12, antibody 2B12 and antibody 12B11. Further of such antibodies are provided in PCT/EP2013/069302 and PCT/EP2013/069293, both filed 17 Sep. 2013, the disclosures of which antibodies are incorporated herein by reference. AZ158 binds human KIR3DL2 as well as human KIR3DL1 and KIR3DS1 polypeptides, 2B12 and bind selectively to KIR3DL2 and do not bind KIR3DL1 (or KIR3DS1). Antibody AZ158 can be used, for example, as therapeutic agent administered to an individual for the elimination of a KIR3DL2 expressing target, e.g. by induction of ADCC and/or CDC. Antibody 2B12, 19H12 and 12B111 are also suitable for use as therapeutic agent administered to an individual for the elimination of a KIR3DL2-expressing target cells. 19H12 and 12B11 as well as other antibodies disclosed in PCT/EP2013/069293 are capable of being internalized into cells via KIR3DL2 and can be used advantageously as an antibody-drug conjugate. 2B12 and other antibodies disclosed in PCT/EP2013/069302 do no induce any KIR3DL2 internalization into tumor cells, thereby providing advantageous use when effector cell mediated activity is sought, e.g. for depleting antibodies that induce ADCC. PCT/EP2015/055224 discloses humanized antibodies. One such antibody that bind human KIR3DL2 and is suitable for use as therapeutic agent administered to an individual for the elimination of a KIR3DL2-expressing target cells is known under the commercial name LACUTAMAB (see WHO Drug Information, Vol. 32, No. 4, 2018).

The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, neutrophils, and eosinophils.

As used herein, “T cells” refers to a sub-population of lymphocytes that mature in the thymus, and which display, among other molecules T cell receptors on their surface. T cells can be identified by virtue of certain characteristics and biological properties, such as the expression of specific surface antigens including the TCR, CD4 or CD8, optionally CD4 and IL-23R, the ability of certain T cells to kill tumor or infected cells, the ability of certain T cells to activate other cells of the immune system, and the ability to release protein molecules called cytokines that stimulate or inhibit the immune response.

### Producing Diagnostic Antibodies

The antibodies or antibodies fragments thereof specifically bind to KIR3DL2 particularly in fixed samples such as formalin-fixed paraffin-embedded (FFPE) tissue sections. The antibodies can specifically bind to their target antigen in a biological sample (e.g. a FFPE section) comprising KIR3DL2-expressing cells that have been prepared as a paraffin-embedded cell pellet. The ability of the antibodies to specifically bind KIR3DL2 in paraffin-embedded tissue sections makes them useful for numerous applications, in particular for detecting the KIR3DL2 or KIR3DL2-expressing cells (e.g. cancer cells) and levels or distribution of KIR3DL2 or KIR3DL2-expressing cells for diagnostic or therapeutic purposes, as described herein. In certain embodiments, the antibodies or antibodies fragments thereof are used to determine the presence or level of KIR3DL2-expressing cells in or near cancerous tissue in a sample (e.g. biopsy) taken from an individual, for example an individual having a cancer. Optionally further, in one embodiment, if KIR3DL2 is detected in the tissue sample, KIR3DL2-expressing cells are determined to be present. Optionally, in another embodiment, if KIR3DL2 is detected (optionally if a predetermined level of KIR3DL2 is detected) in the tissue sample, the individual is determined to be suited for treatment with a therapeutic antibody that binds the KIR3DL2.

The detection of the binding of the antibody to target antigen can be performed in any of a number of ways. For example, the antibody can be directly labeled with a detectable moiety, e.g., a luminescent compound such as a fluorescent moiety, or with a radioactive compound, with gold, with biotin (which allows subsequent, amplified binding to avidin, e.g., avidin-AP), or with an enzyme such as alkaline phosphatase (AP) or horseradish peroxidase (HRP). In an alternative and preferred embodiment, the binding of the antibody to the target antigen in the sample is assessed indirectly, for example by using a secondary antibody that binds to the primary anti-target antigen antibody and that itself is labeled, preferably with an enzyme such as horseradish peroxidase (HRP) or alkaline phosphatase (AP); however, it will be appreciated that the secondary antibodies can be labeled or detected using any suitable method. In a preferred embodiment, an amplification system is used to enhance the signal provided by the secondary antibody, for example the EnVision system in which the secondary antibodies are bound to a polymer (e.g., dextran) that is bound to many copies of a detectable compound or enzyme such as HRP or AP (see, e.g., Wiedorn et al. (2001) The Journal of Histochemistry & Cytochemistry, Volume 49(9): 1067-1071; Kammerer et al., (2001) Journal of Histochemistry and Cytochemistry, Vol. 49, 623-630; the entire disclosures of which are herein incorporated by reference).

KIR3DL2 polypeptide or one or more immunogenic fragments thereof can be used as immunogens to raise antibodies, and the antibodies can recognize epitopes within KIR3DL2 polypeptide on paraffin-embedded samples as described herein. The antibodies according to the disclosure can recognize an epitope present on the extracellular (i.e. they are accessible to antibodies present outside of the cell) or intracellular domain of KIR3DL2 polypeptide. Preferably, the recognized epitopes are present on the intracellular domain of KIR3DL2 polypeptide. Such epitope present on the intracellular domain of KIR3DL2 polypeptide are accessible to antibodies in FFPE sample as tissues are cut into sections before being into contact with said antibody. In one aspect, the epitope is the epitope specifically recognized in a paraffin-embedded cell pellet sample by antibody.

The antibodies of this invention may be produced by a variety of techniques known in the art. Typically, they are produced by immunization of a non-human animal, preferably a rabbit, with an immunogen comprising KIR3DL2 polypeptide, preferably a human KIR3DL2 polypeptide. The polypeptide may comprise the full length sequence of the human polypeptide, or a fragment or derivative thereof, typically an immunogenic fragment, i.e., a portion of the polypeptide comprising an epitope exposed on the surface of cells expressing a KIR3DL2 polypeptide. Such fragments typically contain at least about 7 consecutive amino acids of the mature polypeptide sequence, even more preferably at least about 10 consecutive amino acids thereof. Fragments typically are essentially derived from the extra-cellular domain of KIR3DL2 receptor. Such fragment can typically be designed by using tools such as IHC Peptide Profiler™ technology (BIOTEM Corp.). In a preferred embodiment, the immunization is realized by injection of a pool of at least two immunogenic fragment derived from KIR3DL2 polypeptide and obtained by such afore-mentioned tools, preferably a pool of at least three immunogenic fragment. In one embodiment, immunogenic fragments derived from KIR3DL2 polypeptide are defined by the sequences disclosed in the following table.

TABLE 4

| Peptide | SEQ ID NO: 23 | CEHFFLHREGISEDPSRLVG |
|---|---|---|
| 4484_1 | | |
| Peptide | SEQ ID NO: 24 | CTPLTDTSVYTELPNAEPRS |
| 4484_3 | | |
| Peptide | SEQ ID NO: 25 | CPRAPQSGLEGVF |
| 4484_4 | | |

The step of immunizing a non-human mammal with an KIR3DL2 polypeptide or immunogenic fragments thereof may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, NY (1988), the entire disclosure of which is herein incorporated by reference). The immunogen is suspended or dissolved in a buffer, optionally with an adjuvant, such as complete or incomplete Freund's adjuvant. Methods for determining the amount of immunogen, types of buffers and amounts of adjuvant are well known to those of skill in the art and are not limiting in any way on the present invention. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 21, optionally with an adjuvant such as incomplete Freund's adjuvant. The recall injections are performed intravenously and may be repeated for several consecutive days. This is followed by a booster injection at day 35, either intravenously or intraperitoneally, typically without adjuvant. This protocol results in the production of antigen-specific antibody-producing B cells after about 45 days. Other protocols may also be used as long as they result in the production of B cells expressing an antibody directed to the antigen used in immunization.

In an alternate embodiment, lymphocytes from a non-immunized non-human mammal are isolated, grown in vitro, and then exposed to the immunogen in cell culture. The lymphocytes are then harvested and the fusion step described below is carried out.

B cells from the splenocytes can be isolated from the immunized non-human mammal total RNA can be extracted and quantified in order to build scFv libraries. Constructions of such libraries are common for one skilled in the art (e.g. Lennard S et al. (2002) Standard Protocols for the Construction of scFv Libraries. In Antibody Phage Display. Methods in Molecular Biology™, vol 178.). In details, RNA coding variable domains of the γ chain and κ/λ light chains can be retro-amplified with specific primer sets, respectively. VH and VL PCR products of amplification can be separately pooled and cloned in a backup vector in order to generate two distinct sub-libraries (one for the heavy and one for the light chains). VL fragments are cloned in a phagemid vector, then the VH fragments were inserted into the vector containing the VL repertoire. Typically, the vector format VH/VL-6His (HHHHHH)-FLAG (DYKDDDDK) is suitable for such constructions. Typically, a vector suitable for building a scFv library in such disclosure is M13K07 phage. Once constructed, the scFv library can be screened through common methods such as phage display. To this purpose, the phage-displayed scFvs library can subjected to several pannings using different strategies, against KIR3DL2 polypeptide or fragments thereof. Anti-KIR3DL2 peptide isolated clones DNA can be extracted then sequenced. Such sequence can be inserted into suitable vectors to produce corresponding scFv. Suitable expressing vectors or expressing system are part of the common knowledges. For instance, E. coli strain could be transformed and used for producing such scFv. The reactivity of such scFv against KIR3DL2 polypeptides can be assess to select the best clones.

According to an optional embodiment, the DNA encoding an antibody that binds an epitope present on KIR3DL2 polypeptides isolated from the scFv library is placed in an appropriate expression vector for transfection into an appropriate host cell. The host cell is then used for the recombinant production of the antibody, or variants thereof, such as a humanized version of that monoclonal antibody, active fragments of the antibody, chimeric antibodies comprising the antigen recognition portion of the antibody, or versions comprising a detectable moiety.

DNA encoding the KIR3DL2-specific antibodies of the invention, can be readily isolated and sequenced using conventional procedures (e.g., by using oligonucleotide probes that are capable of binding specifically to genes encoding the heavy and light chains of antibodies). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. As described elsewhere in the present specification, such DNA sequences can be modified for any of a large number of purposes, e.g., for humanizing antibodies, producing fragments or derivatives, or for modifying the sequence of the antibody, e.g., in the antigen binding site in order to optimize the binding specificity of the antibody.

According to one embodiment, provided is an isolated polypeptide comprising or consisting of the amino acid sequence of SEQ ID NO: 24. Alternatively, the isolated polypeptide according to the disclosure can be modified or, fused to one or more heterologous polypeptides or comprised in another polypeptide (e.g. a polypeptide comprising one or more non-KIR3DL2 amino acid sequences). Provided is also an antibody or antibody fragment thereof that binds to said polypeptide comprising or consisting of the amino acid sequence of SEQ ID NO: 24. Binding of the antibody or antibody fragment thereof according to the disclosure to said polypeptide comprising or consisting of the amino acid sequence of SEQ ID NO: 24 can be assessed by any methods known by one-skilled-in-the-art (e.g. ELISA, BIACORE). In a preferred embodiment, such binding is assessed by an ELISA test. ELISA (Enzyme-linked Immunosorbent Assays) test is a simple method allowing the detection of the binding between an antibody and another antigen (e.g. a peptide). As an example, detection by ELISA test of the binding of the antibody or antibody fragment thereof according to the disclosure to said polypeptide comprising or consisting of the amino acid sequence of SEQ ID NO: 24 can comprise the steps of (i) coating a microtiter plate wells with said polypeptide comprising or consisting of the amino acid sequence of SEQ ID NO: 24, (ii) blocking all unbound sites of the plate (e.g. by using BSA) to avoid false positive results, (iii) providing the antibody or antibody fragment thereof (e.g. a soluble scFv) to the wells, (iv) providing a secondary antibody specific of the first antibody or antibody fragment thereof, said antibody being conjugated to an enzyme, (v) providing a substrate suitable to react with the enzyme to produce a colored product, indicating the binding of the antibody or antibody fragment thereof to the polypeptide. Optionally, the polypeptide comprising to the amino acid sequence of SEQ ID NO:24 can be bound on a solid support to proceed to the ELISA test. Such polypeptides can for instance be fused to a linker polypeptide bound to a solid support.

The disclosure provides a method of detection or screening based on formalin-fixed paraffin-embedded sample (FFPE cell pellets) that reveal KIR3DL2 epitopes present following formaldehyde or formalin treatment. Accordingly, in one aspect, the disclosure provides a monoclonal antibody that specifically binds KIR3DL2 polypeptide-expressing cells (e.g. cells made to express KIR3DL2) in a sample preserved as a paraffin-embedded cell pellet, and deparaffinized prior to analysis. Optionally the antibody is further characterized by not or insignificantly binding to KIR3DL2-negative cells (cells that do not express KIR3DL2) in a paraffin-embedded cell pellet. Optionally the antibody is further characterized by binding to KIR3DL2 polypeptide-expressing cells in paraffin-embedded tissue sections.

In one aspect the disclosure provides a monoclonal antibody or an antibody fragment thereof that specifically binds a human KIR3DL2 polypeptide, wherein said antibody specifically binds to said KIR3DL2 polypeptide in a biological sample that has been fixed using formaldehyde (e.g. formalin, paraformaldehyde). Formalin fixation can be used in particular the preparation of paraffin embedded tissue sections which can then be deparaffinized and analyzed for presence of a marker of interest, e.g. KIR3DL2 polypeptide.

In one aspect, provided is a monoclonal antibody that specifically binds a human KIR3DL2 polypeptide expressed by a cell that has been preserved in paraffin, e.g. a cell that has been preserved as a paraffin-embedded cell pellet. Optionally, the cells are pelleted, formaldehyde treated (e.g. formaldehyde, formalin, paraformaldehyde) and then paraffin embedded. Optionally, the cell that expresses the human KIR3DL2 polypeptide is in a biological sample that has been deparaffinized prior to analysis.

In one aspect, the antibodies bind an antigenic determinant present on KIR3DL2 in a FFPE cell pellet sample. The residues bound by the antibody can be specified as being present on the surface of the KIR3DL2 polypeptide, optionally further in a KIR3DL2 polypeptide expressed by a cell.

In one embodiment, provided is a method of making or testing an antibody or antibody fragment thereof capable of binding to a KIR3DL2 polypeptide in a biological sample, comprising determining whether the antibody or antibody fragment binds an amino acid sequence selected from the group consisting of CEHFFLHREGISEDPSRLVG (SEQ ID NO: 23), CTPLTDTSVYTELPNAEPRS (SEQ ID NO: 24) and CPRAPQSGLEGVF (SEQ ID NO: 25).

In one embodiment, provided is a method of making or testing an antibody or antibody fragment thereof capable of binding to a KIR3DL2 polypeptide in a biological sample, comprising determining whether the antibody or antibody fragment binds the same epitope on KIR3DL2 as antibody having a VH of SEQ ID NO: 21 and a VL of SEQ ID NO: 22.

In one embodiment, provided is a method of making an antibody or antibody fragment thereof capable of binding to a KIR3DL2 polypeptide in a biological sample, comprising immunizing a non-human mammal with an amino acid sequence selected from the group consisting of CEHFFLHREGISEDPSRLVG (SEQ ID NO: 23), CTPLTDTSVYTELPNAEPRS (SEQ ID NO: 24) and CPRAPQSGLEGVF (SEQ ID NO: 25), isolating antibodies from the mammal that bind to KIR3DL2, and optionally further assessing and/or selecting antibodies therefrom for their ability to bind to a KIR3DL2 polypeptide in a biological sample (e.g. an FFPE sample). In one embodiment, provided is a method of making an antibody or antibody fragment thereof capable of binding to a KIR3DL2 polypeptide in a biological sample, comprising providing a plurality of antibodies and assessing and/or selecting antibody(ies) for the capacity to bind an amino acid sequence selected from the group consisting of CEHFFLHREGISEDPSRLVG (SEQ ID NO: 23), CTPLTDTSVYTELPNAEPRS (SEQ ID NO: 24) and CPRAPQSGLEGVF (SEQ ID NO: 25), and optionally further assessing whether the antibody or antibody fragment thereof capable of binding to a KIR3DL2 polypeptide in a biological sample (e.g. an FFPE sample). In one embodiment, provided is an antibody or antibody fragment obtained by a method of making an antibody or antibody fragment of the disclosure.

In one aspect, provided is an antibody or an antibody fragment thereof capable of specifically binding to a KIR3DL2 polypeptide comprising a heavy chain variable region (VH) comprising the amino acid sequence of SEQ ID NO: 21, and a light chain variable region (VL) comprising the acid sequence of SEQ ID NO: 22. (See table 5 below).

TABLE 5

| | | |
|---|---|---|
| VH | SEQ ID NO: | QSLEESGGRLVTPGTPLTLTCTVSGFSLSTYAMSWVRQAPGKGL |
| | 21 | EWIGIIGASGNTWYASWAKGRFTISKTSTTVGLKITSPTTEDTATYF |
| | | CARFWAGYPSNAAATVSGMDPWGPGTLVTVSS |
| VL | SEQ ID NO: | ELVLTQSPSLSASLDTTARLACTLSTGYSVGSYGIGWYQQVPGRP |
| | 22 | PRYLLTYHTEEIKHQGSGVPTRFSGSKDTSENTAVLSISGLQPEDE |
| | | ADYYCATAHGSGSSFHVVFGGGTQLTVT |

In one aspect, the antibody capable of specifically binding to a KIR3DL2 polypeptide comprises a heavy chain having at least about 80% sequence identity (e.g., at least about 85%, 90%, 95%, 97%, 98%, 99% or more identity) to the heavy chain having the amino acid sequence of SEQ ID NO: 21.

In one aspect, the antibody capable of specifically binding to a KIR3DL2 polypeptide comprises a light chain having at least about 80% sequence identity (e.g., at least about 85%, 90%, 95%, 97%, 98%, 99% or more identity) to the light chain having the amino acid sequence of SEQ ID NO: 22.

In any aspect, an antibody capable of specifically binding to a KIR3DL2 polypeptide can be specified as comprising VH and VL frameworks (e.g., FR1, FR2, FR3 and FR4) of human origin.

Antibodies according to the present invention can comprise the antigen binding region (e.g. light chains VH/VL) as defined herein fused to an immunoglobulin constant region of the IgG type, optionally a constant region, optionally a IgG1, IgG2, IgG3 or IgG4 isotype, optionally further comprising an amino acid substitution to reduce effector function (binding to Fcγ receptors). In one embodiment, the antigen binging region as defined hereinabove can be fused to a rabbit immunoglobulin constant region of the igG type.

In some embodiments, provided is an antibody comprising the heavy chain CDR 1, 2 and 3 (HCDR1, HCDR2, HCDR3) of the VH amino acid sequence of SEQ ID NO: 21, and the light chain CDR 1, 2 and 3 (LDR1, LDR2, LCDR3) of the VL amino acid sequence of SEQ ID NO: 22. Optionally, CDRs are determined according to Kabat, Chothia, Abm or IMGT numbering schemes.

Provided in one aspect is an antibody comprising (i) a heavy chain comprising CDR 1, 2 and 3 (HCDR1, HCDR2, HCDR3) having a sequence of SEQ ID NO: 03 (HDR1), SEQ ID NO: 06 (HCDR2) and SEQ ID NO: 09 (HCDR3), and (ii) a light chain comprising CDR 1, 2 and 3 (LCDR1, LDR2, LCDR3) having a sequence of SEQ ID NO: 12 (LWDRA), SEQ ID NO: 15 (LCDR2), and SEQ ID NO: 18 (LCDR3). (See tables 6 and 7 below).

TABLE 6

| | CDR | HCDR1 | | HCDR2 | | HCDR3 | |
|---|---|---|---|---|---|---|---|
| | def- | SEQ | | SEQ | | SEQ | |
| mAb | inition | ID | Sequence | ID | Sequence | ID | Sequence |
| P3- | Kabat | NO: | TYAMS | NO: | IIGASGNTWYASWAK | NO: | FWAGYPSN |
| R4D- | | 3 | | 6 | G | 9 | AAATVSGM |
| H5 | | | | | | | DP |
| | Chotia | NO: | GFSLSTY | NO: | ASG | NO: | WAGYPSNA |
| | | 4 | | 7 | | 10 | AATVSGMD |
| | IMGT | NO: | GFSLSTYA | NO: | IGASGNT | NO: | ARFWAGYP |
| | | 5 | | 8 | | 11 | SNAAATVS |
| | | | | | | | GMDP |

TABLE 7

| | CDR | LCDR1 | | LCDR2 | | LCDR3 | |
|---|---|---|---|---|---|---|---|
| | def- | SEQ | | SEQ | | SEQ | |
| mAb | inition | ID | Sequence | ID | Sequence | ID | Sequence |
| P3- | Kabat | NO: | TLSTGYSV | NO: | YHTEEIKHQGS | NO: | ATAHGSGS |
| R4D- | | 12 | GSYGIG | 15 | | 18 | SFHVV |
| H5 | Chotia | NO: | LSTGYSV | NO: | YHTEEIK | NO: | AHGSGSSF |
| | | 13 | GSYG | 16 | | 19 | HV |
| | IMGT | NO: | TGYSVGS | NO: | YHTEEIK | NO: | ATAHGSGS |
| | | 14 | YG | 17 | | 20 | SFHVV |

Optionally any one or more of said light or heavy chain CDRs may contain one, two, three, four or five or more amino acid modifications (e.g. substitutions, insertions or deletions).

In one aspect, the antibody capable of specifically binding to a KIR3DL2 polypeptide comprises: a HCDR1 comprising an amino acid sequence: TYAMS (SEQ ID NO: 3), or a sequence of at least 4 contiguous amino acids thereof, optionally wherein one or more of these amino acids may be substituted by a different amino acid; a HCDR2 comprising an amino acid sequence: IIGASGNTWYASWAKG (SEQ ID NO: 6), or a sequence of at least 4, 5, 6, 7, 8, 9, 10, 11, 12, 13, 14 or 15 contiguous amino acids thereof, optionally wherein one or more of these amino acids may be substituted by a different amino acid; a HCDR3 comprising an amino acid sequence: FWAGYPSNAAATVSGMDP (SEQ ID NO: 9), or a sequence of at least 4, 5, 6, 7, 8, 9, 10, 11, 12, 13, 14, 15, 16, 17 or 18 contiguous amino acids thereof, optionally wherein one or more of these amino acids may be substituted by a different amino acid; a LCDR1 comprising an amino acid sequence: TLSTGYSVGSYGIG (SEQ ID NO: 12), or a sequence of at least 4, 5, 6, 7, 8, 9, 10, 11, 12 or 13 contiguous amino acids thereof, optionally wherein one or more of these amino acids may be substituted by a different amino acid; a LCDR2 region comprising an amino acid sequence: YHTEEIKHQGS (SEQ ID NO: 15) or a sequence of at least 4, 5, 6, 7, 8, 9 or 10 contiguous amino acids thereof, optionally wherein one or more of these amino acids may be substituted by a different amino acid; and/or a LCDR3 region comprising an amino acid sequence: ATAHGSGSSFHVV (SEQ ID NO: 18), or a sequence of at least 4, 5, 6, 7, 8, 9, 10, 11 or 12 contiguous amino acids thereof, optionally wherein one or more of these amino acids may be deleted or substituted by a different amino acid.

A further object of the present invention also encompasses function-conservative variants of the antibodies capable of specifically binding to a KIR3DL2 polypeptide of the present disclosure. “Function-conservative variants” are those in which a given amino acid residue in a protein (e.g. an antibody or antibody fragment) has been changed without altering the overall conformation and function of the protein, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity with the antibody capable of specifically binding to a KIR3DL2 polypeptide as defined hereinabove as determined by BLAST or FASTA algorithms, preferably at least 75%, more preferably at least 85%, still preferably at least 90%, and even more preferably at least 95%, and which has the same or substantially similar properties or functions as the antibodies capable of specifically binding to a KIR3DL2 polypeptide as defined hereinabove.

The specified heavy chain, light chain, variable region and CDR sequences may comprise sequence modifications, e.g. a substitution (1, 2, 3, 4, 5, 6, 7, 8 or more sequence modifications). In one embodiment, an amino acid sequence comprises one, two, three or more amino acid substitutions, where the residue substituted is a residue present in a sequence of human origin. In one embodiment the substitution is a conservative modification. A conservative sequence modification refers to an amino acid modification that does not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are typically those in which an amino acid residue is replaced with an amino acid residue having a side chain with similar physicochemical properties. Specified amino acid sequences may comprise one, two, three, four or more amino acid insertions, deletions or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g. threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the properties set forth herein) using the assays described herein.

In one embodiment, the antibodies of the invention are antibody fragments that retain their binding and/or functional properties. Fragments and derivatives of antibodies of this invention (which are encompassed by the term “antibody” or “antibodies” as used in this application, unless otherwise stated or clearly contradicted by context), preferably an anti-KIR3DL2 antibody, can be produced by techniques that are known in the art. “Fragments” comprise a portion of the intact antibody, generally the antigen binding site or variable region. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments.

### Preparation and Staining of FFPE Samples

The present antibodies have the particular property of being able to efficiently and specifically bind to polypeptides (e.g. KIR3DL2 polypeptides) present in fixed tissue or cell samples. Various methods of preparing and using such tissue preparations are well known in the art, and any suitable method or type of preparation can be used. The antibodies are further capable of binding their target antigen in samples in which therapeutic (e.g. function neutralizing) antibodies were present at or prior to fixation.

The FFPE material in a biological sample from an individual is typically a tissue. FFPE tissue is a piece of tissue which is first separated from a specimen animal (e.g., human individual) by dissection or biopsy. Then, this tissue is fixed in order to prevent it from decaying or degenerating and to permit one to examine it clearly under a microscope for histological, pathological or cytological studies. Fixation is the process by which the tissue is immobilized, killed and preserved for the purpose of staining and viewing it under a microscope. Post-fixation processing makes tissue permeable to staining reagents and cross-links its macromolecules so that they are stabilized and locked in position. This fixed tissue is then embedded in the wax to allow it to be cut into thin sections and be stained with hematoxylin and eosin stain. After that, microtoming is done by cutting fine sections to study stain with antibodies under microscope.

It will be appreciated, for example, that the present antibodies can be used with different suitable fixed cell or tissue preparations, and different particular fixation or embedding methods used. For example, while the most common formaldehyde-based fixation procedure involves formalin (e.g., 10%), alternative methods such as paraformaldehyde (PFA), Bouin solution (formalin/picric acid), alcohol, zinc-based solutions (for one example, see, e.g., Lykidis et al., (2007) Nucleic Acids Research, 2007, 1-10, the entire disclosure of which is herein incorporated in its entirety), and others (see, e.g., the HOPE method, Pathology Research and Practice, Volume 197, Number 12, December 2001, pp. 823-826(4), the entire disclosure of which is herein incorporated by reference). Similarly, while paraffin is preferred, other materials can be used for embedding as well, e.g., polyester wax, polyethylene glycol based formulas, glycol methacrylates, JB-4 plastics, and others. For review of methods for preparing and using tissue preparations, see, e.g., Gillespie et al., (2002) Am J Pathol. 2002 February; 160(2): 449-457; Fischer et al. CSH Protocols; 2008; Renshaw (2007), Immunohistochemistry: Methods Express Series; Bancroft (2007) Theory and Practice of Histological Techniques; and PCT patent publication no. WO06074392; the entire disclosures of which are herein incorporated by reference).

In one embodiment of the invention, the FFPE tissue is a human tissue (e.g. tumor tissue, tumor-adjacent tissue, normal tissue) in which expression of KIR3DL2 is sought to be investigated. For example, the FFPE tissue can be any human tumor tissue in which expression of KIR3DL2 is sought to be investigated. The tumor may be, for example, tumor of the squamous epithelium, bladder, stomach, kidneys, head and neck, skin, breast, gastrointestinal tract, colon, oesophagus, ovary, cervix, thyroid, intestine, liver, brain, pancreas, prostate, urogenital tract, lymphatic system, stomach, larynx and/or lung. The FFPE tissue may, for example, derived from a blood sample. In one embodiment, when KIR3DL2 is detected, the FFPE tissue may be a tumor or tumor-adjacent tissue obtained from in individual who has received treatment with an anti-KIR3DL2 antibody, e.g., who has undergone or is undergoing a course of therapy with such antibody.

The antibody (e.g. anti-KIR3DL2 antibody) is incubated with the FFPE material for detection of KIR3DL2 polypeptides. The term incubation step involves the contacting of the FFPE material with the antibody of the invention for a distinct period, which depends on the kind of material, antibody and/or antigen. The incubation process also depends on various other parameters, e.g. the sensitivity of detection, which optimization follows routine procedures known to those skilled in the art. Adding chemical solutions and/or applying physical procedures, e.g. impact of heat, can improve the accessibility of the target structures in the sample. Specific incubation products are formed as result of the incubation.

Suitable tests for the detection of formed antibody/antigen complexes are known to those skilled in the art or can be easily designed as a matter of routine. Many different types of assays are known, examples of which are set forth below. Although the assay may be any assay suitable to use anti-KIR3DL2 mAb binding to detect and/or quantify KIR3DL2 expression, the latter is preferably determined by means of substances specifically interacting with the primary anti-KIR3DL2 antibody.

Thus, for example, the sample (tissue or cells) to be examined is obtained by biopsy from a biological fluid, tumor tissue or from a healthy tissue, and sections (e.g., 3 mm thick or less) and fixed using formalin or an equivalent fixation method (see supra). The time of fixation depends on the application, but can range from several hours to 24 or more hours. Following fixation, the tissue is embedded in paraffin (or equivalent material), and very thin sections (e.g., 5 microns) are cut in a microtome and then mounted onto, preferably coated, slides. The slides are then dried, e.g., air dried.

Fixed and embedded tissue sections on slides can be dried and stored indefinitely. For immunohistochemistry, the slides are deparaffinized and then rehydrated. For example, they are subjected to a series of washes with, initially, xylene, and then xylene with ethanol, and then with decreasing percentages of ethanol in water.

Before antibody staining, the tissues can be subjected to an antigen retrieval step, e.g., enzymatic or heat-based, in order to break methane bridges that form during fixation and which can mask epitopes. In a preferred embodiment, a treatment in boiling 10 mM citrate buffer, pH 6, is used.

Once the slides have been rehydrated and antigen retrieval has been ideally performed, they can be incubated with the primary antibody. First, the slides are washed with, e.g., TBS, and then, following a blocking step with, e.g., serum/BSA, the antibody can be applied. The concentration of the antibody will depend on its form (e.g., purified), its affinity, the tissue sample used, but a suitable concentration is, e.g., 1-10 μg/ml. In one embodiment, the concentration used is 10 μg/ml. The time of incubation can vary as well, but an overnight incubation is typically suitable. Following a post-antibody washing step in, e.g., TBS, the slides are then processed for detection of antibody binding.

The detection method used will depend on the antibody, tissue, etc. used, and can for example involve detection of a luminescent or otherwise visible or detectable moiety conjugated to the primary antibody, or through the use of detectable secondary antibodies. Methods of antibody detection are well known in the art and are taught, e.g., in Harlow et al., Antibodies: A Laboratory Manual, Cold Spring Harbor Laboratory Press, 1st edition (Dec. 1, 1988); Fischer et al. CSH Protocols; 2008; Renshaw (2007), Immunohistochemistry: Methods Express Series; Bancroft (2007) Theory and Practice of Histological Techniques; PCT patent publication no. WO06074392; the entire disclosure of each of which is herein incorporated in its entirety.

Many direct or indirect detection methods are known and may be adapted for use. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the protein is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine.

Indirect labels include various enzymes well known in the art, such as horseradish peroxidase (HRP), alkaline phosphatase (AP), β-galactosidase, urease and the like. The covalent linkage of an anti-3DL2 antibody to an enzyme may be performed by different methods, such as the coupling with glutaraldehyde. Both, the enzyme and the antibody are interlinked with glutaraldehyde via free amino groups, and the by-products of networked enzymes and antibodies are removed. In another method, the enzyme is coupled to the antibody via sugar residues if it is a glycoprotein, such as peroxidase. The enzyme is oxidized by sodium periodate and directly interlinked with amino groups of the antibody. Other enzyme containing carbohydrates can also be coupled to the antibody in this manner. Enzyme coupling may also be performed by interlinking the amino groups of the antibody with free thiol groups of an enzyme, such as β-galactosidase, using a heterobifunctional linker, such as succinimidyl 6-(N-maleimido) hexanoate. The horseradish-peroxidase detection system can be used, for example, with the chromogenic substrate tetramethylbenzidine (TMB), which yields a soluble product in the presence of hydrogen peroxide that is detectable at 450 nm. The alkaline phosphatase detection system can be used with the chromogenic substrate β-nitrophenyl phosphate, for example, which yields a soluble product readily detectable at 405 nm. Similarly, the β-galactosidase detection system can be used with the chromogenic substrate o-nitrophenyl-β-D-galactopyranoxide (ONPG), which yields a soluble product detectable at 410 nm. A urease detection system can be used with a substrate, such as urea-bromocresol purple.

In one embodiment, the binding of the primary antibody is detected by binding a labeled secondary antibody, preferably a secondary antibody covalently linked to an enzyme such as HRP or AP. In a particularly preferred embodiment, the signal generated by binding of the secondary antibody is amplified using any of a number of methods for amplification of antibody detection. For example, the EnVision method can be used, (see, e.g., U.S. Pat. No. 5,543,332 and European Patent no. 594,772; Kammerer et al., (2001) Journal of Histochemistry and Cytochemistry, Vol. 49, 623-630; Wiedorn et al. (2001) The Journal of Histochemistry & Cytochemistry, Volume 49(9): 1067-1071; the entire disclosures of which are herein incorporated by reference), in which the secondary antibodies are linked to a polymer (e.g., dextran) that is itself linked to many copies of AP or HRP.

### Uses of Antibodies in Diagnostics, Prognostics and Therapy and Methods Thereof

The antibodies of the invention are particularly effective at detecting KIR3DL2 polypeptide within biological samples. In a preferred embodiment, the antibodies of the invention are particularly effective at detecting KIR3DL2 polypeptide within tissue samples. In a further embodiment, the antibodies of the invention are particularly effective at detecting KIR3DL2 polypeptide within fixed tissue samples. In a preferred embodiment, the antibodies of the invention are particularly effective at detecting KIR3DL2 polypeptide within formalin-fixed paraffin-embedded tissue samples (FFPE), without non-specific staining on tissues or cells that do not express target antigen polypeptides. The antibodies will therefore have advantages for use in the study, evaluation, diagnosis, prognosis and/or monitoring in diseases where detection of and/or localization of KIR3DL2 polypeptide and/or KIR3DL2-expressing cells is of interest.

Accordingly, provided are methods of detecting, diagnosing, or monitoring cancer in an individual, the method comprising the steps of contacting (e.g. in vitro) cancerous cells with an anti-KIR3DL2 antibody or antibody fragment thereof of the disclosure and detecting the cancerous cell-associated KIR3DL2 polypeptide. In related embodiments the diagnostic method will comprise immunohistochemistry (IHC). In certain embodiments, the biological sample is a tissue sample. In a further embodiment, the biological sample is a fixed tissue sample. In a preferred embodiment, the biological sample is a formalin fixed and/or paraffin embedded tissue sample. Those of skill in the art will further appreciate that such KIR3DL2 detection agents may be labeled or associated with effectors, markers or reporters as and detected using any one of a number of standard imaging techniques. In other embodiments the anti-KIR3DL2 antibody will not be directly labelled and will be detected using a secondary agent that is detectable (e.g., a labelled anti-rabbit antibody). In certain embodiments, the present disclosure provides a method for identifying or selecting an individual for administration of a therapy (e.g. a chemotherapy, an immunotherapy) comprising diagnosing an individual using any of the anti-KIR3DL2 compositions and detection methods of the invention, and tailoring a course of therapy based on the outcome. The disclosure further provides a method for selecting individuals having a KIR3DL2-expressing cancer for administering an agent that enhances an anti-tumor response regimen (e.g. a chemotherapeutic agent, an immunotherapeutic agent, such as an anti-KIR3DL2 monoclonal antibody), for preventing or treating a cancer. In certain embodiments, the disclosure provides a method for monitoring (e.g. the efficacy of) a therapy (e.g. a therapeutic anti-KIR3DL2 antibody) for preventing or treating a cancer.

The antibodies described herein can be used for the detection, preferably in vitro, of the presence KIR3DL2-expressing cells, for example cancerous cells (e.g. cancerous CD4 T cells, cancerous CD8 T cells). Such a method will typically involve contacting a biological sample from an individual with an antibody according to the disclosure and detecting the formation of immunological complexes resulting from the immunological reaction between the antibody and the biological sample. In certain embodiments, the biological sample is a tissue sample. In a further embodiment, the biological sample is a fixed tissue sample. In a preferred embodiment, the biological sample is a formalin fixed and/or paraffin embedded tissue sample. The complex can be detected directly by labelling the antibody according to the disclosure or indirectly by adding a molecule which reveals the presence of the antibody according to the invention (secondary antibody, etc.). For example, labelling can be accomplished by coupling the antibody with radioactive or fluorescent tags. These methods are well known to those skilled in the art. Accordingly, the invention also relates to the use of an antibody according to the disclosure for preparing a diagnostic composition that can be used for detecting the presence of KIR3DL2-expressing cells (e.g., cancerous cells, cancerous CD4 T cells, cancerous CD8 T cells), optionally for detecting the presence of a pathology where KIR3DL2-expressing cells are present, optionally for characterizing a cancer or other pathology, in vivo or in vitro.

In some embodiments, the antibodies of the disclosure will be useful for predicting cancer progression. A cancer prognosis, a prognostic for cancer or cancer progression comprises providing the forecast or prediction of (prognostic for) any one or more of the following: duration of survival of a subject susceptible to or diagnosed with a cancer, duration of recurrence-free survival, duration of progression free survival of a subject susceptible to or diagnosed with a cancer, response rate to treatment in a subject or group of subjects susceptible to or diagnosed with a cancer, and/or duration of response, degree of response, or survival following treatment in a subject. Exemplary survival endpoints include for example TTP (time to progression), PFS (progression free survival), DOR (duration of response), and OS (overall survival). Generally, disease progression and responses can be determined according to standard tumor response criteria conventions, for example according to “Response Evaluation Criteria in Solid Tumors” (RECIST) v1.1 as detailed by Eisenhauer, E A, et al, New response evaluation criteria in solid tumours: Revised RECIST guideline (version 1.1), Eur J Cancer 2009:45:228-247; the disclosure of which is incorporated by reference herein.

In some aspects, a biological sample from an individual having a cancer can be characterized or assessed using an antibody disclosed herein to assess KIR3DL2 polypeptide and/or KIR3DL2-expressing cells in a biological sample. In certain embodiments, the biological sample is a tissue sample. In a further embodiment, the biological sample is a fixed tissue sample. In a preferred embodiment, the biological sample is a formalin fixed and/or paraffin embedded tissue sample. In one embodiment, the individual has not yet been treated with an agent (e.g. therapeutic antibody) that binds KIR3DL2 polypeptides and enhance cytotoxicity toward the KIR3DL2-expressing cells (e.g. through ADCC). Alternatively, the individual has undergone or is undergoing a course of therapy with a therapeutic antibody that binds KIR3DL2 polypeptide, e.g. an anti-KIR3DL2 monoclonal antibody such as lacutamab.

The methods of the disclosure can be useful to determine whether the individual has a cancer characterized by KIR3DL2-expressing cells. Such methods can be useful to determine and/or provide an optimal course of therapy for the individual, as well as to monitor disease in the individual.

In one embodiment, the disclosure provides a method for the detection of KIR3DL2-expressing cells in an individual having a cancer, the method comprising providing a biological sample from the individual, optionally wherein the sample comprises tumor tissue or cancerous cells, and detecting KIR3DL2 polypeptide in said sample using a monoclonal antibody of the disclosure. In certain embodiments, the biological sample is a tissue sample. In a further embodiment, the biological sample is a fixed tissue sample. In a preferred embodiment, the biological sample is a formalin fixed and/or paraffin embedded tissue sample. In one embodiment, a detection of KIR3DL2 polypeptide indicates the presence of KIR3DL2-expressing cells in the tissue, optionally wherein the KIR3DL2-expressing cells are cancerous cells.

In one embodiment, the individual has undergone or is undergoing a course of therapy with a chemotherapeutic agent.

In one embodiment, the individual is eligible (e.g., the individual is a candidate or potential candidate) for a course of therapy with an agent (e.g. a therapeutic antibody) that binds a KIR3DL2 polypeptide.

In one embodiment, the individual has undergone or is undergoing a course of therapy with a therapeutic antibody that specifically binds KIR3DL2 polypeptides and/or KIR3DL2-expressing cells.

In one embodiment, a cancer or tumor characterized by KIR3DL2 and/or KIR3DL2-expressing cells (or an individual having such cancer or tumor) can be identified as being suitable for (e.g. benefitting from) treatment with a chemotherapeutic agent or an immunotherapeutic agent. For example, the immunotherapeutic agent may be an agent that directly or indirectly acts on KIR3DL2-expressing cells by inducing cytotoxicity (e.g. through ADCC). Such agents may be useful to treat individuals having cancer or tumor tissue characterized by detectable and/or elevated levels of KIR3DL2 expression.

In one embodiment, the disclosure provides an in vitro method for the diagnosis, prognosis, monitoring and/or characterization of a cancer in an individual in need thereof, the method comprising providing a biological sample from an individual, and detecting KIR3DL2 polypeptide (e.g. KIR3DL2-expressing cells) in the sample using a monoclonal antibody that specifically binds to a human KIR3DL2 polypeptide, preferably in a fixed tissue sample, optionally a paraffin-embedded tissue sample, wherein a detection of KIR3DL2 polypeptide indicates that the individual is amenable to (e.g. benefitting from) treatment with a chemotherapeutic agent or an immunotherapeutic agent. The immunotherapeutic agent may be an agent that directly or indirectly acts on KIR3DL2-expressing cells by inducing cytotoxicity (e.g. through ADCC). Such agents may be useful to treat individuals having cancer or tumor tissue characterized by detectable and/or elevated levels of KIR3DL2 expression. In one embodiment, the biological sample is a tissue sample. In one embodiment, the biological sample is a fixed-tissue sample, preferably a chemically fixed tissue sample. In one embodiment, the biological sample is a formalin-fixed paraffin embedded (FFPE) tissue.

In one embodiment, the individual has undergone or is undergoing a course of therapy with a therapeutic antibody that binds KIR3DL2 polypeptide (e.g. KIR3DL2-expressing cells) and induces cytotoxicity (e.g. through ADCC) such as Lacutamab. Biological samples from the individual can be assessed for the expression and/or level of KIR3DL2. If KIR3DL2 is detectable, then the individual may be deemed suitable for continued or further treatment with the therapeutic antibody that binds KIR3DL2 polypeptide (e.g. KIR3DL2-expressing cells) and induces cytotoxicity (e.g. through ADCC), and/or for treatment with an additional or different therapeutic agent. Accordingly, in one embodiment, the disclosure provides an in vitro method for the diagnosis, prognosis, monitoring and/or characterization of a cancer in an individual in need thereof, the method comprising providing a paraffin-embedded biological sample from an individual who has been treated with a therapeutic antibody that binds KIR3DL2 polypeptide (e.g. KIR3DL2-expressing cells) and induces cytotoxicity (e.g. through ADCC) such as Lacutamab, and detecting KIR3DL2 polypeptide (e.g. KIR3DL2-expressing cells) in the sample using a monoclonal antibody that specifically binds to a human KIR3DL2 polypeptide in a fixed tissue sample, optionally a paraffin-embedded tissue sample, wherein a detection of KIR3DL2 polypeptide indicates that the individual is suitable for (e.g. benefitting from) treatment with a chemotherapeutic agent or an immunotherapeutic agent.

In one embodiment, an individual whose cancerous cells or tumor tissue is characterized by expressing KIR3DL2 polypeptide can be treated with an anti-cancer agent, e.g. a chemotherapeutic agent, an immunotherapeutic agent, an agent that binds KIR3DL2 polypeptide (e.g. KIR3DL2-expressing cells) and enhances cytotoxicity (e.g. through ADCC) against KIR3DL2-expressing cells such as Lacutamab. Such agents may be useful to treat individuals having tumors or tumor tissue characterized by detectable and/or elevated levels KIR3DL2 expression.

In one embodiment, the disclosure provides a method for the treatment or prevention of a cancer in an individual in need thereof, the method comprising:
(i) providing a biological sample from an individual, detecting KIR3DL2-expressing cells in said sample,
(ii) upon a determination that the biological sample KIR3DL2-expressing cells, optionally at a level that is increased compared to a reference level, administering to the individual an immunotherapeutic agent.

In certain embodiments, the biological sample is a tissue sample. In a further embodiment, the biological sample is a fixed tissue sample. In a preferred embodiment, the biological sample is a formalin fixed and/or paraffin embedded tissue sample.

In one embodiment, the disclosure provides a method for the treatment or prevention of a cancer in an individual in need thereof, the method comprising:
(i) providing a biological sample from an individual, detecting KIR3DL2-expressing cells in said sample, using an antibody of the disclosure, and
(ii) upon a determination that the biological sample KIR3DL2-expressing cells, optionally at a level that is increased compared to a reference level, administering to the individual an immunotherapeutic agent.

In certain embodiments, the biological sample is a tissue sample. In a further embodiment, the biological sample is a fixed tissue sample. In a preferred embodiment, the biological sample is a formalin fixed and/or paraffin embedded tissue sample.

In one embodiment, the disclosure provides a method for the treatment or prevention of a cancer in an individual in need thereof, the method comprising:
(i) providing a biological sample from an individual, detecting KIR3DL2-expressing cells in said sample, using an antibody of the disclosure, and
(ii) upon a determination that the biological sample KIR3DL2-expressing cells, optionally at a level that is increased compared to a reference level, administering to the individual an immunotherapeutic agent that binds KIR3DL2 polypeptide (e.g. KIR3DL2-expressing cells) and enhances cytotoxicity (e.g. through ADCC) against KIR3DL2-expressing cells such as Lacutamab.

In certain embodiments, the biological sample is a tissue sample. In a further embodiment, the biological sample is a fixed tissue sample. In a preferred embodiment, the biological sample is a formalin fixed and/or paraffin embedded tissue sample.

In any aspect, detecting KIR3DL2 polypeptide in sample using an antibody can comprise the steps of contacting a biological sample from an individual with the antibody and detecting the formation of immunological complexes resulting from the immunological reaction between the antibody and the biological sample. In certain embodiments, the biological sample is a tissue sample. In a further embodiment, the biological sample is a fixed tissue sample. In a preferred embodiment, the biological sample is a formalin fixed and/or paraffin embedded tissue sample.

Agents that bind KIR3DL2 (e.g. KIR3DL2 at the surface of a tumor cell) may be a depleting anti-KIR3DL2 antibody (e.g., a KIR3DL2-binding antibody or antibody fragment, optionally fused with a cytotoxic agent such as a toxin), a chimeric antigen receptor (e.g. a CAR-T cell receptor) comprising a KIR3DL2-binding antibody fragment, an effector cell (e.g. NK or T cell) expressing at its surface the chimeric antigen receptor), a polypeptide fused to an Fc domain, an immunoadhesin, etc., that binds KIR3DL2. Examples of antibody agents (therapeutic antibodies) are disclosed in PCT publication WO2014/044686.

The antibody is optionally characterized by an EC50 in 51 Cr-release assay for HuT78 tumor lysis by PBMC from healthy volunteers, of less than 100 ng/ml, optionally between 1 and 100 ng/ml, optionally between 1 and 50 ng/ml, optionally between 25 and 75 ng/ml, optionally about 50 ng/ml. The antibody is optionally characterized by an EC50 in 51Cr-release assay for HuT78 tumor lysis by PBMC from healthy volunteers comparable to that of an anti-KIR3DL2 antibody disclosed herein (e.g., having an EC50 that is lower or within 1-log or 0.5-log of the EC50 of that of an antibody having a VH of SEQ ID NO: 3 and a VL of SEQ ID NO: 4, comprising an Fc domain of wild type or modified human IgG1 isotype, and that mediates ADCC.

In one embodiment, the therapeutic anti-KIR3DL2 antibody used in accordance with the disclosure is or comprises the heavy and light chain amino acid sequence of lacutamab (see WHO Drug Information, Vol. 32, No. 4, 2018). Lacutamab is a humanized antibody having the Kabat heavy chain CDR1, 2 and 3 of the heavy chain variable region of SEQ ID NO: 26 and the Kabat light chain CDR1, 2 and 3 of the light chain variable region of SEQ ID NO: 27 (See table 8 below). CDRs can be determined by a suitable numbering scheme, e.g. Kabat numbering. In one embodiment, lacutamab can be characterized as comprising a heavy chain variable region comprising an amino acid sequence of SEQ ID NO: 26 and a light chain variable region comprising an amino acid sequence of SEQ ID NO: 27.

TABLE 8

| | | |
|---|---|---|
| Lacutamab, | SEQ ID NO: 26 | QIQLVQSGSELKKPGASVKVSCKASGYTFTTAGMQWV |
| VH | | RQAPGQGLEWIGWINSHSGVPKYAEDFKGRFVFSLDTS |
| | | VSTAYLQISSLKAEDTAVYFCARGGDEGVMDYWGQGT |
| | | TVTVSS |
| Lacutamab, | SEQ ID NO: 27 | DIQMTQSPSFLSASVGDRVTITCKASQDVSTAVAWYQQ |
| VL | | KPGQPPKLLIYWTSTRHTGVPDRFSGSGSGTDYTLTISS |
| | | LQAEDVAVYYCQQHYSTPWTFGGGTKVEIK |

In one embodiment, lacutamab can be characterized as comprising a heavy chain comprising an amino acid sequence of SEQ ID NO: 28 and a light comprising an amino acid sequence of SEQ ID NO: 29 (See table 9 below).

TABLE 9

| | | |
|---|---|---|
| Lacutamab, | SEQ ID NO: 28 | QIQLVQSGSELKKPGASVKVSCKASGYTFTTAGMQWV |
| VH | | RQAPGQGLEWIGWINSHSGVPKYAEDFKGRFVFSLDTS |
| | | VSTAYLQISSLKAEDTAVYFCARGGDEGVMDYWGQGT |
| | | TVTVSSASTKGPSVFPLAPSSKSTSGGTAALGCLVKDYF |
| | | PEPVTVSWNSGALTSGVHTFPAVLQSSGLYSLSSVVTV |
| | | PSSSLGTQTYICNVNHKPSNTKVDKRVEPKSCDKTHTC |
| | | PPCPAPELLGGPDVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALPAPEEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| Lacutamab, | SEQ ID NO: 29 | DIQMTQSPSFLSASVGDRVTITCKASQDVSTAVAWYQQ |
| VL | | KPGQPPKLLIYWTSTRHTGVPDRFSGSGSGTDYTLTISS |
| | | LQAEDVAVYYCQQHYSTPWTFGGGTKVEIKRTVAAPSV |
| | | FIFPPSDEQLKSGTASVVCLLNNFYPREAKVQWKVDNA |
| | | LQSGNSQESVTEQDSKDSTYSLSSTLTLSKADYEKHKV |
| | | YACEVTHQGLSSPVTKSFNRGEC |

In one embodiment, the anti-KIR3DL2 therapeutic antibody comprises the heavy and light chain CDRs of antibody 11E1, 8C7, 3C12 and/or 6E1 disclosed in POT publications WO2014/044681 or WO2014/044686.

Also provided are diagnostic or prognostic kits, e.g., for cancer, comprising an antibody or an antibody fragment thereof according to the disclosure. Optionally the kit comprises antibody or antibody fragment thereof of the invention and a labelled secondary antibody that specifically recognizes said antibody or antibody fragment thereof of the disclosure. Optionally the kit comprises an antibody of the invention for use as a diagnostic or prognostic, and an immunotherapeutic agent. In one embodiment, the immunotherapeutic agent is an agent that binds KIR3DL2 polypeptide (e.g. KIR3DL2-expressing cells) and enhances cytotoxicity (e.g. through ADCC) against KIR3DL2-expressing cells such as Lacutamab. Said kit can additionally comprise means by which to detect the immunological complex resulting from the immunological reaction between the biological sample and an antibody, in particular reagents enabling the detection of said labelled antibody.

The present methods may be useful in the study, evaluation, diagnosis, prognosis, and/or monitoring of a range of cancers.

Such methods are suitable for detecting, assessing the suitability for treatment and/or treating individuals having a TCL, susceptible to a TCL or having experienced a TCL. In one embodiment, the TCL is an aggressive or advanced TCL (e.g. stage IV, or more generally beyond stage II). In one embodiment, the individual has relapsing or refractory disease. In one embodiment, the individual has a poor prognosis for disease progression (e.g. poor prognosis for survival), has a poor prognosis for response to a therapy, or has progressing or relapsing disease following prior treatment with a prior therapy.

In one embodiment, the TCL is an aggressive T-cell neoplasm. In one embodiment, the TCL is aggressive non-cutaneous TCL. In another embodiment, the TCL is aggressive cutaneous TCL, optionally a primary cutaneous CD4+ small/medium T cell lymphoma or a primary CD8+ small/medium T cell lymphoma. In one embodiment, the TCL is a cutaneous T cell lymphoma (CTCL). In one embodiment, the TCL is a peripheral T cell lymphoma (PTCL), optionally a non-cutaneous PTCL. PTCL and PTCL-NOS may optionally be specified to be diseases other than cutaneous T cell lymphomas.

Cutaneous T-cell lymphoma (CTCL) (see the image below) is a group of lymphoproliferative disorders characterized by localization of neoplastic T lymphocytes to the skin. Collectively, CTCL is classified as a type of non-Hodgkin lymphoma (NHL). The World Health Organization-European Organization for Research and Treatment of Cancer (WHO-EORTC) classification of CTCLs is reported in Willemze et al. (2005) Blood 105:3768-3785. The WHO-EORTC divides CTCL into those with indolent clinical behavior and those with aggressive subtypes. A third category is that of precursor hematologic neoplasms that are not T-cell lymphomas (CD4+/CD56+ hematodermic neoplasm, blastic natural killer (NK)-cell lymphoma or B-cell derived primary cutaneous neoplasms). CTCLs which can have indolent clinical behavior include Mycosis fungoides (MF) and its variants, primary cutaneous CD30+ lymphoproliferative disorder (e.g., primary cutaneous anaplastic large cell lymphoma, lymphomatoid papulosis), subcutaneous panniculitis-like T-cell lymphoma (provisional) and primary cutaneous CD4+ small/medium-sized pleomorphic T-cell lymphoma (provisional). CTCLs with aggressive clinical behavior include Sezary syndrome (SS), Adult T-cell leukemia/lymphoma, Extranodal NK/T-cell lymphoma, nasal type, Primary cutaneous peripheral T-cell lymphoma, unspecified, Primary cutaneous aggressive epidermotropic CD8+ T-cell lymphoma (provisional) and Cutaneous gamma/delta-positive T-cell lymphoma (provisional). The methods disclosed herein can be used to treat each of these conditions.

The most common CTLCs are MF and SS. Their features are reviewed, e.g. in Willemze et al. (2005) Blood 105:3768-3785, the disclosure of which is incorporated herein by reference. In most cases of MF, the diagnosis is reached owing to its clinical features, disease history, and histomorphologic and cytomorphologic findings. An additional diagnostic criterion to distinguish CTCL from inflammatory dermatoses is demonstration of a dominant T-cell clone in skin biopsy specimens by a molecular assay (e.g., Southern blot, polymerase chain reaction (PCR)). Genetic testing may also be considered. Classic mycosis fungoides is divided into three stages: (1) Patch (atrophic or non-atrophic): Nonspecific dermatitis, patches on lower trunk and buttocks; minimal/absent pruritus; (2) Plaque: Intensely pruritic plaques, lymphadenopathy and (3) Tumor: Prone to ulceration. Sezary syndrome is defined by erythroderma and leukemia. Signs and symptoms include edematous skin, lymphadenopathy, palmar and/or plantar hyperkeratosis, alopecia, nail dystrophy, ectropion and hepatosplenomegaly. For a diagnosis of Sezary syndrome, criteria typically include absolute Sezary cell count, immunophenotypic abnormalities, loss of T-cell antigens and/or a T-cell clone in the peripheral blood shown by molecular or cytogenetic methods.

CTCL stages include I, II, Ill and IV, according to TNM classification, and as appropriate, peripheral blood involvement. Peripheral blood involvement with mycosis fungoides or Sezary syndrome (MF/SS) cells is correlated with more advanced skin stage, lymph node and visceral involvement, and shortened survival. MF and SS have a formal staging system proposed by the International Society for Cutaneous Lymphomas (ISCL) and the European Organization of Research and Treatment of Cancer (EORTC). See, Olsen et al., (2007) Blood. 110(6):1713-1722; and Agar et al. (2010) J. Clin. Oncol. 28(31):4730-4739, the disclosures of which are incorporated herein by reference.

In one embodiment, the TCL is a peripheral T cell lymphoma (PTCL), optionally a non-cutaneous PTCL. PTCL and PTCL-NOS may optionally be specified to be diseases other than cutaneous T cell lymphomas Sezary Syndrome and Mycosis fungoides which are considered distinct pathologies. In one embodiment, the PTCL is a nodal (e.g. primarily or predominantly nodal) PTCL. Predominantly nodal PTCLs include, inter alia, PTCL-NOS (Peripheral T-cell lymphomas, not otherwise specified), anaplastic large cell lymphomas (ALCL) and angioimmunoblastic T-cell lymphomas (AITL), For example a PTCL may be an aggressive, non-cutaneous, predominantly nodal PCTL (the disease may additionally have extra-nodal presentation).

In one embodiment, the PTCL is an extranodal (e.g. primarily extranodal) PTCL. For example a PTCL may be an aggressive, non-cutaneous, extranodal PCTL.

In one embodiment, the PTCL is an adult T cell leukemia or lymphoma (ATL), e.g., an HTLV+ATL.

In one embodiment, the PTCL is an extranodal NK-/T-cell lymphoma, nasal type. In one embodiment, the PTCL is an enteropathy-associated T cell lymphoma.

In one embodiment, the PTCL is a hepatosplenic T cell lymphoma, optionally a hepatosplenic as T cell lymphoma, optionally a hepatosplenic γδ T cell lymphoma.

In one embodiment, the PTCL is an anaplastic large cell lymphoma (ALCL), optionally an ALK+ALCL, optionally an ALK−ALCL. ALK+ALCL generally enjoys favorable prognostics using conventional therapy (93% 5 year survival) but ALK−ALCL has poor prognostics (37%).

In one embodiment, the PTCL is an angioimmunoblastic T-cell lymphoma (AITL), optionally a cutaneous AITL, optionally a primary cutaneous CD4+ small/medium T cell lymphoma or a primary CD8+ small/medium T cell lymphoma, optionally a non-cutaneous AITL.

In one embodiment, the PTCL is an intestinal lymphoma, e.g. an intestinal ALCL.

In one embodiment, the PTCL is a T-cell prolymphocytic leukemia.

In one embodiment, a PTCL is a PTCL-NOS (Peripheral T-cell lymphoma, not otherwise specified). PTCL-NOS, also referred to as PCTL-U or PTCL-unspecified, are aggressive lymphomas, mainly of nodal type, but extranodal involvement is common. The majority of nodal cases are CD4+ and CD8−. Most individuals with PTCL-NOS present with nodal involvement; however, a number of extranodal sites may also be involved (e.g., liver, bone marrow, gastrointestinal, skin. Studies generally report a 5-year overall survival of approximately 30%-35% using standard chemotherapy. In the past, a number of definite entities corresponding to recognizable subtypes of T-cell neoplasm, such as Lennert lymphoma, T-zone lymphoma, pleomorphic T-cell lymphoma and T-immunoblastic lymphoma have been described, but evidence that these correspond to distinctive clinicopathologic entities is still lacking. For this reason the recent World Health Organization (WHO) classification of the hematopoietic and lymphoid neoplasms has collected these under the single broad category of PTCL-NOS/U. PTCL-NOS may therefore be specified to exclude certain distinctive clinicopathologic entities such as T-cell prolymphocytic leukemia, ATL/adult T cell leukemia, extranodal NK-/T-cell leukemia nasal type, EATL/enteropathy-type T cell lymphoma, hepatosplenic T-cell lymphoma, subcutaneous panniculitis-like T-cell lymphoma, ALCL/anaplastic large-cell lymphoma, and/or AITL/angioimmunoblastic T cell lymphoma.

PTCL diagnosis criteria can be those of standard medical guidelines, for example, according to the World Health Organization (WHO) classification system (see, e.g., World Health Organization. WHO Classification of Tumours of Haematopoietic and Lymphoid Tissues, 4th ed. Lyon, France: IARC Press, 2008). See also, e.g., Foss et al. (2011) Blood 117:6756-6767, the disclosures of which are incorporated herein by reference.

In one embodiment, a TCL is characterized by tumors or tumor cells that express significant and/or detectable KIR3DL2 polypeptides at their surface. Advantageously, KIR3DL2 expression is determined by a method according to the disclosure.

Also provided is a nucleic acid encoding an antibody or antibody fragment of the disclosure. In one embodiment the nucleic acid is an isolated and/or recombinant (including, e.g., essentially pure) nucleic acid comprising sequences which encode an antibody or antibody fragment of the present disclosure.

Provided is a hybridoma or recombinant host cell producing an antibody fragment of the disclosure. A hybridoma or recombinant host cell of the disclosure can thus comprises a nucleic acid encoding an antibody or antibody fragment of the disclosure.

### EXAMPLES

### Example 1: Generation of a Monoclonal Antibody for KIR3DL2 Immunohistochemistry (IHC) Staining in Formalin-Fixed Paraffin-Embedded (FFPE) Samples

### Rabbit Immunizations

Rabbit immunizations for generating anti-KIR3DL2 antibodies were performed using 4 different strategies:
(1) Immunization of 2 rabbits (rabbits #121 and 122) with native recombinant KIR3DL2 protein
(2) Immunization of 2 rabbits (rabbits #278 and 372) with recombinant extracellular domain of the KIR3DL2 protein treated according to a first IHC-like protocol (IHC-A))
(3) Immunization of 2 rabbits (rabbits #373 and 374) with recombinant extracellular domain of the KIR3DL2 protein treated according to a second IHC-like protocol (IHC-B)
(4) Immunization of 2 rabbits (rabbits #375 and 376) with a pool of 3 peptides (peptide 1: CEHFFLHREGISEDPSRLVG (SEQ ID NO: 23), peptide 3: CTPLTDTSVYTELPNAEPRS (SEQ ID NO: 24) and peptide 4: CPRAPQSGLEGVF (SEQ ID NO: 25)) designed with the IHC Peptide Profiler™ technology (Biotem Corp., France). Peptide 1 is an epitope from the extracellular domain of KIR3DL2 receptors, whereas peptides 3 and 4 are epitopes from the intracellular domain of KIR3DL2 receptors.

Such peptides were produced and conjugates to carrier proteins (Immunogen peptides to KLH and Screening peptides to BSA and/or free).

Rabbits were injected (subcutaneous route) with antigen on day 1, 21, 35. Bleeding was performed on day 45. Sera were screened by ELISA on proteins and peptides and IHC on FFPE tissue microarray.

Rabbits 121 and 122 present similar profiles but according to titers reached rabbit 121 seems to be a better candidate for library construction. In IHC, a good reactivity can be observed with no clear specificity for KIR3DL2. Rabbits 278 and 372 present similar profiles but according to titers reached rabbit 278 seems to be a better candidate for library construction. In IHC, a good reactivity can be observed with no clear specificity for KIR3DL2. Rabbits 373 and 374 present similar profiles but according to titers reached rabbit 374 seems to be a better candidate for library construction. In IHC, a good reactivity can be observed with no clear specificity for KIR3DL2. Rabbits 375 and 376 present similar profiles but according to titers reached rabbit 376 seems to be a better candidate for library construction. In IHC, a good reactivity/specificity can be observed for KIR3DL2.

Altogether, these data led to the selection of 1 animal immunized with the pool of peptides (rabbit #376) and 1 animal immunized with the recombinant extracellular domain of the KIR3DL2 protein treated with the IHC-like procedure B (rabbit #374) that showed strong Ab titers by ELISA and more intense IHC stainings. If the serum obtained after the immunization with the pool of peptides gave staining on KIR3DL2-transfected CHO cells and no staining on non-transfected CHO cells, the serum obtained after immunization with the recombinant extracellular domain of the KIR3DL2 protein treated with the IHC-like procedure B stained both KIR3DL2- and KIR3DL1-transfected cells. The later was selected as a second choice because of potential cross-reactivity against KIR3DL1.

Construction of scFV Library

Splenectomy was performed on selected rabbits. Spleens were treated in the molecular biology laboratory for RNA extraction. Total RNA were then quantified. RNA coding variable domains of the γ chain and κ/λ light chains were retro-amplified with specific primer sets, respectively. The quality of the amplification was controlled by electrophoresis, with a 1% TBE/0.8% agarose-gel. The SmartLadder SF MW-1800-04 (Eurogentec) was used as reference during the electrophoresis. VH and VL PCR products of amplification were separately pooled and cloned in a backup vector in order to generate two distinct sub-libraries (one for the heavy and one for the light chains). The first step of the library construction consisted of the VL fragments cloning in our phagemid vector, and then the VH fragments were inserted into the vector containing the VL repertoire. The Vector format VH/VL-6His (HHHHHH)-FLAG (DYKDDDDK) has been selected for constructions.

The final scFv library constructed from the rabbit immunized with the KIR3DL2 protein treated with the IHC-like procedure B consisted of 2.4×10⁸ independent clones with a full-size insert rate of 87% (by colony-PCR) and was finally packaged in M13K07 phage. The final scFv library constructed from the rabbit immunized with the pool of 3 peptides consisted of 1.7×10⁸ independent clones with a full-size insert rate of 94% (by colony-PCR) and was finally packaged in M13K07 phage. After selection of reactive clones and sequence analysis of the candidates, 24 clones were produced in E. coli as soluble scFV and purified. Among those candidates, 16 came from the rabbit immunization with the recombinant extracellular domain of the KIR3DL2 protein treated with the IHC-like procedure B (rabbit #374) and 8 came from the rabbit immunization with the pool of peptides (rabbit #376).

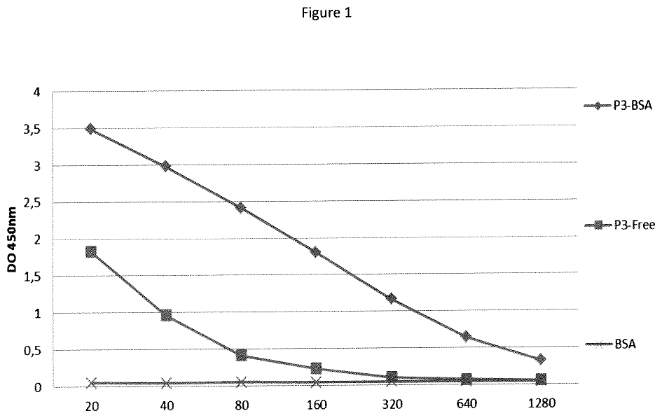

Reactivity of the scFV was assessed by ELISA for these candidates. Strong reactivity was observed against the KIR3DL2 protein but not against the three peptides 1, 3 and 4 for the 16 scFv retrieved from the rabbit immunization with the recombinant extracellular domain of the KIR3DL2 protein treated with the IHC-like procedure B. Among the 8 scFv retrieved from the rabbit immunization with the pool of peptides, the two anti-peptide 1 scFv (P1-R7A-C11 and P1-R7A-G1) presented a high reactivity against the targeted peptide 1 conjugated to BSA or free. Reactivity was also observed against the extracellular domain of the KIR3DL2. The six anti-peptide 3 scFv showed a reactivity against the peptide 3 (stronger for the peptide conjugated to BSA compared with the free one) and no reactivity was observed against BSA. The reactivity of clone P3-R4D-H5 against the peptide 3 free or conjugated to BSA evaluated by ELISA is presented on FIG. 1. It appears that clone P3-R4D-H5 have a stronger reactivity against the peptide 3 coupled with BSA than the peptide 3 free.

Reactivity of those 24 candidates was then assessed by IHC using KIR3DL2- and KIR3DL1-transfected CHO cells. The 16 clones identified from the immunization with the KIR3DL2 protein treated with the IHC-like procedure B (rabbit #374) gave a strong signal on both KIR3DL2- and KIR3DL1-transfected CHO cells.

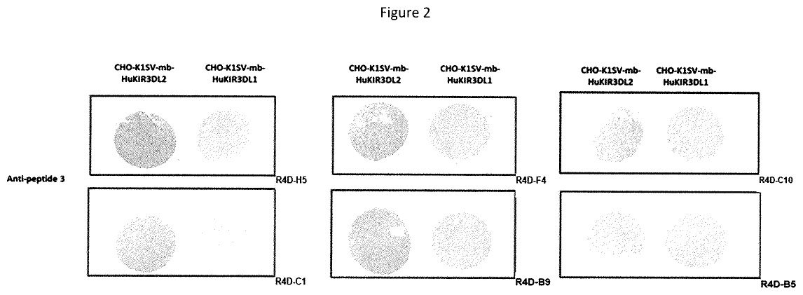

Among, the 8 candidates selected from the rabbit immunization with the pool of peptides (rabbit #376), two were specific for the peptide 1 (P1-R7A-G1 and P1-R7A-C11) and 6 were specific for the peptide 3 (P3-R4D-F4, P3-R4D-C1, P3-R4D-H5, P3-R4D-C10, P3-R4D-B5 and P3-R4D-B9). Both anti-peptide 1 clones gave a strong staining on KIR3DL2-transfected cells and a lower signal on KIR3DL1-transfected cells. According to the ELISA data, the clone P1-R7A-C11 seems to be a better candidate than clone P1-R7A-G1 and the former candidate was selected. As shown on FIG. 2, all the anti-peptide 3 candidates presented a strong reactivity on KIR3DL2-transfected cells and a lower reactivity on KIR3DL1-tranfected cells. The strongest differential staining on KIR3DL2-transfected cells versus KIR3DL1-transfected cells was obtained with clone P3-R4D-H5, demonstrating its specificity to KIR3DL2 polypeptides. Differential staining was also obtained with clones P3-R4D-C10, P3-R4D-B5 and P3-R4D-B9. Because they gave the strongest differential staining on KIR3DL2-transfected cells versus KIR3DL1-transfected cells, the following clones were selected: P3-R4D-H5, P3-R4D-C10, P3-R4D-B5 and P3-R4D-B9.

In conclusion, based on ELISA and IHC data, P1-R7A-C11, P3-R4D-H5, P3-R4D-C10, P3-R4D-B5 and P3-R4D-B9 were selected and produced in a rabbit IgG format.

### Reformatting and IgG Production

Selected scFv were reformatted in full rabbit IgG antibodies: P1-R7A-C11 (rabbit IgG/K), P3-R4D-H5 (rabbit IgG/A), P3-R4D-C10 (rabbit IgG/A), P3-R4D-B5 (rabbit IgG/A) and P3-R4D-B9 (rabbit IgG/A). Sequences encoding the variable domain of heavy chain (VH) and the variable domain of light chain (VL) (presented in the table below) were optimized for expression in mammalian cells and synthetized. The corresponding synthetic genes were cloned in a vector system that contains the rabbit constant regions of IgG heavy chain and lambda or kappa light chain. Once validated by sequencing, vectors were amplified for the preparation of low-endotoxin plasmid DNA.

TABLE 10

| Clone | VH | VL |
|---|---|---|
| P3-R4D- | SEQ ID NO: 21 | SEQ ID NO: 22 |
| H5 | QSLEESGGRLVTPGTPLTLTCTV | ELVLTQSPSLSASLDTTARLACTL |
| | SGFSLSTYAMSWVRQAPGKGLE | STGYSVGSYGIGWYQQVPGRPP |
| | WIGIIGASGNTWYASWAKGRFTIS | RYLLTYHTEEIKHQGSGVPTRFS |
| | KTSTTVGLKITSPTTEDTATYFCA | GSKDTSENTAVLSISGLQPEDEA |
| | RFWAGYPSNAAATVSGMDPWG | DYYCATAHGSGSSFHVVFGGGT |
| | PGTLVTVSS | QLTVT |

Transient transfection was performed at Biotem using chinese hamster ovary cells. Supernatants were harvested on the last culture day (before purification) and antibody titers were measured using ForteBio Protein A biosensors. The supernatants were finally purified by protein A affinity chromatography.

### Anti-KIR3DL2 Antibody Candidate Selection for IHC on FFPE Samples

IHC tests were performed on a Ventana Benchmark Ultra automated slide stainer. This stainer is widely used by pathologists and compatible with the development of a companion diagnostic.

First, the selected clones P1-R7A-C11, P3-R4D-H5, P3-R4D-C10, P3-R4D-B5 and P3-R4D-B9 in a rabbit IgG format were tested to assess their specificity on FFPE cell pellet and normal skin in a tissue microarray format. Two clones (P3-R4D-H5 and P1-R7A-C11) were selected as being the more specific Abs for KIR3DL2 after IHC evaluation and were kept for additional tests. These 2 clones were tested at several concentrations (10, 7.5, 5 and 2.5 μg/mL) on FFPE cell pellets in a tissue microarray format that also included normal skin and FFPE human tissues (2 lymph nodes, colon, liver and CTCL). Compared with the clone P1-R7A-C11, the clone P3-R4D-H5 gave a lower unspecific staining on KIR3DL2 negative cells and on FFPE tissues. It also gave a stronger membranous staining intensity on HuT 78 cells with endogenous KIR3DL2 expression compared to that obtained on CHO-mb-HuKIR3DL1 or CHO cells. Clone P3-R4D-H5 gave the best results on FFPE samples.

### Example 2: Use of KIR3DL2 Specific Antibody in IHC Staining of Cell Pellets

KIR3DL2 Staining, on FFPE Cell Pellets, with Clone P3-R4D-H5

A test on CHO-mb-HuKIR3DL2 and HuT 78 ATCC TIB-161 cells was performed because they overexpressed KIR3DL2. After 7 days of culture (3 passages), CHO (KIR3DL2 negative cells) were mixed with CHO-mb-HuKIR3DL2 cells to generate 8 mixes of KIR3DL2 negative and positive cells at different ratio. After 17 days of culture (7 passages), Raji and HuT cells were mixed by serial dilution to generate 8 mixes of KIR3DL2 negative and positive cells at different ratio. FFPE cell pellets were prepared with 20 million of cells/pellet. Cell lines were fixed for 1 hour in formalin. Then, cells were washed in PBS 1× and resuspended in melted histogel. After histogel solidification at +5±3° C., cell pellets were placed in standard cassettes and dehydrated with a tissue processor. Next, the cell pellets were embedded in paraffin. After paraffin solidification, the FFPE cell pellet blocks were stored at room temperature (RT). Sections were dewaxed for 30 minutes at 72° C. and an epitope retrieval step was performed with an epitope retrieval buffer 20 minutes at +100° C. The sections were incubated for 20 minutes with the primary antibody (clone P3-R4D-H5 or isotype control). Then, the sections were rinsed, incubated 8 minutes with a post-primary Ab (rabbit anti-mouse) and then incubated 8 minutes with the horseradish peroxidase (HRP) polymer (anti-rabbit-HRP). The revelation of the staining was performed with 3, 3′-diaminobenzidine (DAB) for 10 minutes.

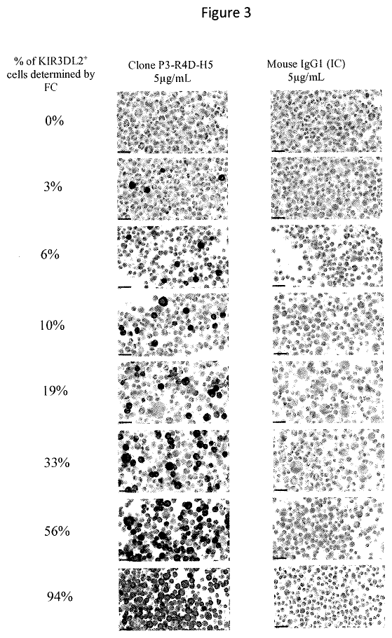

IHC staining was performed with the LEICA BOND RX on 5 sections/block of the 8 CHO/CHO-mb-HuKIR3DL2 mixed FFPE cell pellets using the anti-KIR3DL2 clone P3-R4D-H5 at 5 μg/mL and on 1 section/block of the same FFPE cell pellets using the IC (isotype control) at the same concentration. As shown in FIG. 3, the presence of very low number (until 2.75% determined by flow cytometry) of KIR3DL2+ cells was detectable. As expected, the number of cells stained with the anti-KIR3DL2 antibody clone P3-R4D-H5 increased together with the percentage of KIR3DL2-transfected cells in the pellets. No staining was observed with the isotype control.

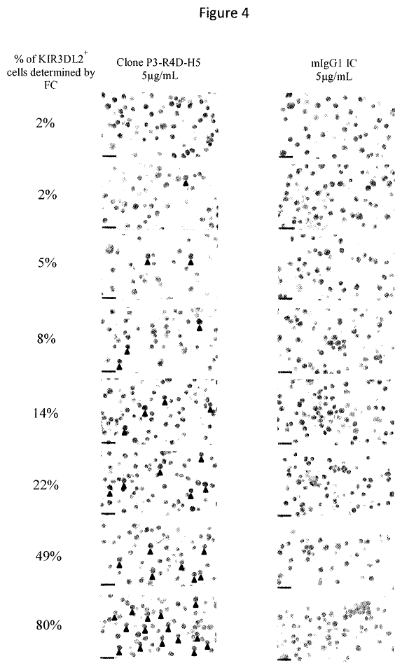

Furthermore, IHC staining was performed with the LEICA BOND RX on 5 sections/block of 8 Raji (human KIR3DL2 negative cells)/HuT (HuT 78, ATCC reference TIB-161, human KIR3DL2 positive cells) mixed FFPE cell pellets using the anti-KIR3DL2 clone P3-R4D-H5 at 5 μg/mL and on 1 section/block of the same pellets using the isotype control at the same concentration. As shown in FIG. 4, here again the presence of very low number (2.42% determined by flow cytometry) of KIR3DL2+ cells was detectable. As expected, the number of cells stained with the anti-KIR3DL2 antibody clone P3-R4D-H5 increased together with the percentage of HuT cells in the pellets. No staining was seen with the isotype control. Altogether, these data on mixed pellets with either transfected cells (CHO-mb-HuKIR3DL2) and cells with endogenous expression (HuT) suggest that the clone P3-R4D-H5 is a specific anti-KIR3DL2 antibody for IHC on FFPE samples. This antibody allows the discrimination of cells with endogenous KIR3DL2 expression and cells without KIR3DL2 expression, with expected percentage values and even when positive cells are present at very low frequency.

### Comparison of Clones P3-R4D-H5 and 12B11 KIR3DL2 Staining

The same type of above-mentioned experiments was performed on mixed frozen pellets and on FFPE cell pellets with the best known tissue staining anti-KIR3DL2 antibody, clone 12B11. The preparation of FFPE cell pellets was the same as described above. Different unmasking and amplification conditions was performed (e.g. buffer, buffer pH, addition of tyramide-biotine). For frozen samples, sections were rehydrated, incubated 10 minutes with 0.3% of H2O2, rinsed three times with PBS 1× squeeze bottle and incubated 30 minutes with protein block. Then, protein block was removed and sections incubated for 1 hour with the primary antibody at room temperature (Anti-KIR3DL2 12B11 antibody or mouse IgG1 isotype control). The sections were rinsed three times for 5 minutes in PBS 1× and then incubated for 30 minutes with an HRP-coupled secondary Ab at RT (EnVision kit from Dako). Then, the sections were rinsed three times for 5 minutes in PBS 1×. Finally, the revelation of the staining was performed with DAB for 5 minutes. For the IHC staining on frozen samples with the LEICA BOND RX, the protocol was the same than for the one used for the FFPE samples without the dewax and the epitope retrieval steps.

Clone P3-R4D-H5 allowed staining of the cell line with endogenous KIR3DL2 expression (HuT cells) on FFPE pellet sections. Clone 12B111 (reference antibody for KIR3DL2 tissue staining) was able to stain the HuT cell line in frozen samples after protocol optimisation. However, further analysis by digital pathology showed that staining was less accurate for the frozen samples stained with the clone 12B111, with a staining of poorer quality compared to that observed for FFPE cell pellet samples. In conclusion, anti-KIR3DL2 clones P3-R4D-H5 and 12B111 for IHC on FFPE and frozen samples, respectively, are KIR3DL2-specific antibodies and allow detection of low numbers of KIR3DL2 expressing cells. However, the quality of staining on FFPE sample is better than that quality of staining with the frozen sample.

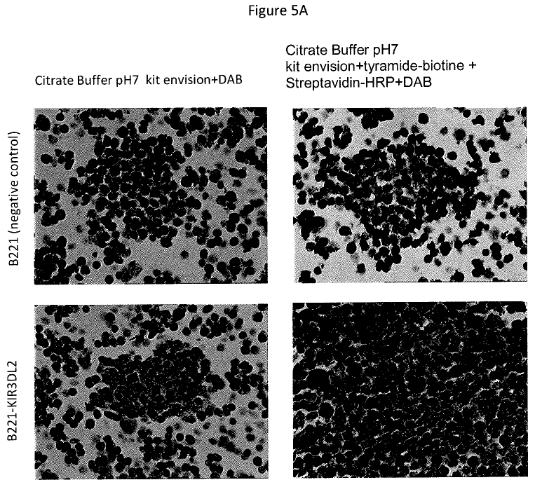

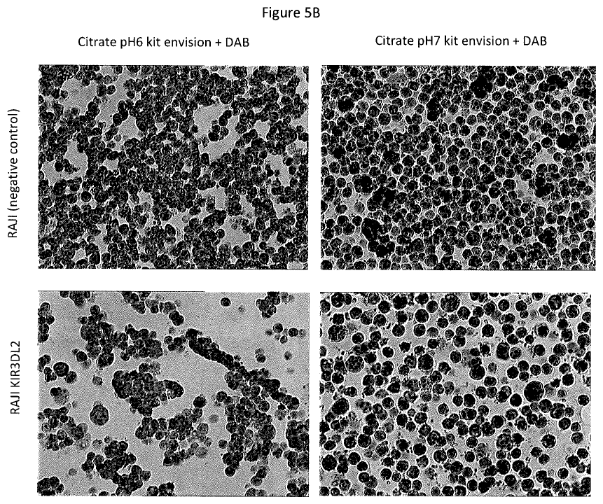

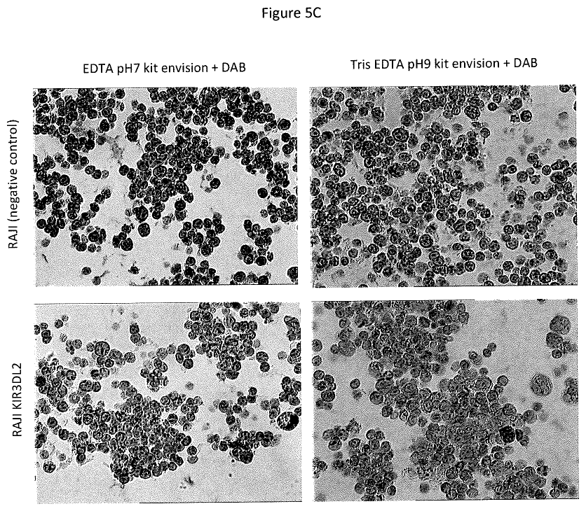

As shown on FIGS. 5A, B and C, an IHC with clone 12B111 Ab (reference antibody for KIR3DL2 tissue staining on frozen sample) under several condition does not allow the revelation of KIR3DL2 on FFPE cell pellets containing KIR3DL2 positive cells. Clone 12B11 can therefore not be used in KIR3DL2 IHC staining on FFPE samples.

### Specificity of Clone P3-R4D-H5 Against KIR3DL2 Versus KIR3DL1 in IHC

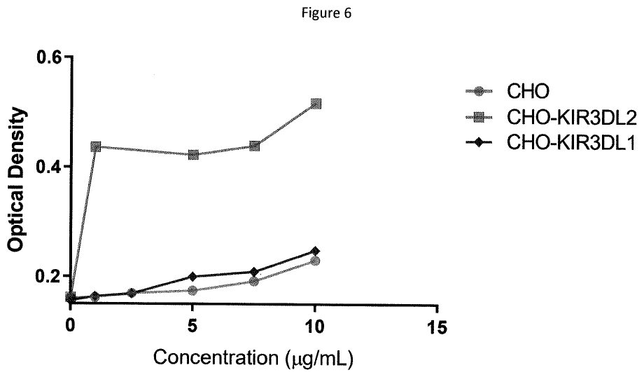

The same type of above-mentioned experiments (See R3DL2 staining, on FFPE cell pellets, with clone P3-R4D-H5) was performed on FFPE cell pellets consisting of CHOcells (CHO) that are human KIR3DL2 and KIR3DL1 negative cells; CHO-mb-HuKIR3DL1 cells (CHO-KIR3DL1) that are human KIR3DL1 positive and KIR3DL2 negative cells; and CHO-mb-HuKIR3DL2 cells (CHO-KIR3DL2) that Human KIR3DL2 positive and KIR3DL1 negative cells. After IHC staining performed using anti-KIR3DL2 antibody clone P3-R4D-H5 at several concentrations, optical densities of FFPE sections were determined by HistoQuantif. As shown on FIG. 6, anti-KIR3DL2 clone P3-R4D-H5 binds to KIR3DL2-expressing cells, whereas no substantial binding on KIR3DL1-expressing cells was observed. Clone P3-R4D-H5 is thus specific to KIR3DL2 polypeptides in FFPE samples.

### Example 3: Use of KIR3DL2 Specific Antibody in IHC Staining of Cell Pellets Prepared from Biopsies

### Staining of CTCL Individual Biopsies

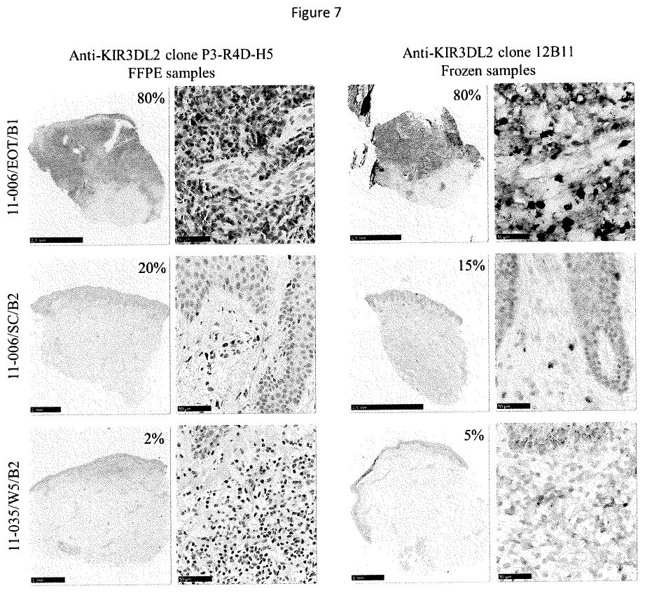

Clones P3-R4D-H5 and 12B111 were used to stain FFPE and matched frozen CTCL biopsies respectively. Staining evaluation on FFPE and frozen sections of biopsies from individual suffering of Mycosis Fungoide or Sezary syndrome was performed by a pathologist on scanned slides. For each FFPE block, 3 μm-thick sections were prepared, deposited on superfrost glass slides and dried at least an hour at +45+/−3° C. in a ventilated oven. Representative images of KIR3DL2 staining on FFPE and frozen sections are shown in FIG. 7. Percentage of KIR3DL2+ cells among mononuclear cells was estimated in a semi quantitative way by a pathologist. Percentages of KIR3DL2+ cells among mononuclear cells were estimated by the pathologist on FFPE CTCL samples stained with the anti-KIR3DL2 clone P3-R4D-H5 and on matched frozen samples stained with the anti-KIR3DL2 clone 12B11.

By comparing the percentage of KIR3DL2+ cells estimated with the clone 12B11 and the clone P3-R4D-H5 on matched CTCL biopsies, it was observed that the clone P3-R4D-H5 usually gave higher percentages of stained cells. This reflects the fact that staining evaluation on frozen samples is more difficult and gave poorer accuracy of that the quality of the frozen samples was poor for those biopsies (frozen samples are more sensitive to temperature changes compared with FFPE samples).

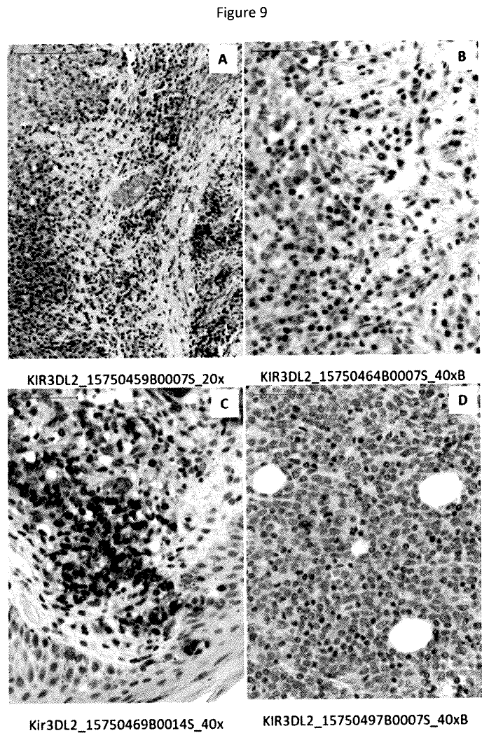

Additionally, further IHC stainings of Mycosis Fungoides tumor samples (CTCL) with the anti-KIR3DL2 clone P3-R4D-H5 according to the method presented above are shown in FIGS. 9A, 9B, 9C and 9D. FIGS. 9A and 9C exhibit tumor samples with a high expression of KIR3DL2 (strong signal), whereas FIG. 9B exhibits a weak positive tumor sample. FIG. 9D shows a recurrent Mycosis Fungoides tumor sample which encompass regions with a strong KIR3DL2 positivity and regions almost KIR3DL2 negative.

### Staining of PTCL Individual Biopsies

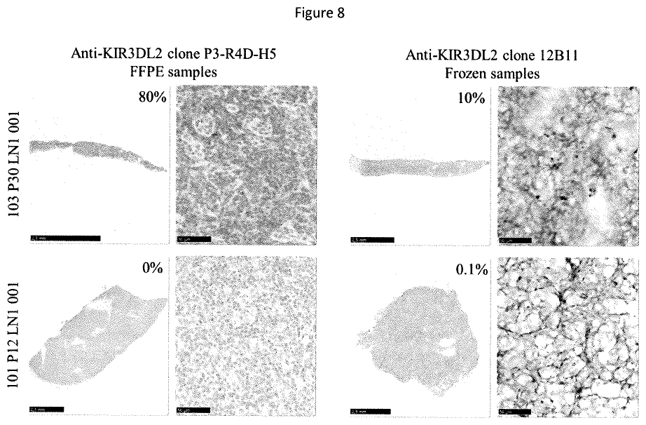

Clones P3-R4D-H5 and 12B11 were used to stain FFPE and match frozen PTCL biopsies respectively. Staining evaluation on FFPE and frozen sections of biopsies from individual suffering of PTCL was performed by a pathologist on scanned slides. Representative images of KIR3DL2 staining on FFPE and frozen sections are shown in FIG. 8. Percentages of KIR3DL2+ cells among mononuclear cells were estimated by the pathologists on frozen PTCL samples stained with the anti-KIR3DL2 clone 12B11 and on matched FFPE samples stained with the anti-KIR3DL2 clone P3-R4D-H5 or isotype control.

Results shows that clone 12B11 and clone P3-R4D-H5 allow the staining of KIR3DL2+ cells on frozen and FFPE PTCL samples, respectively.

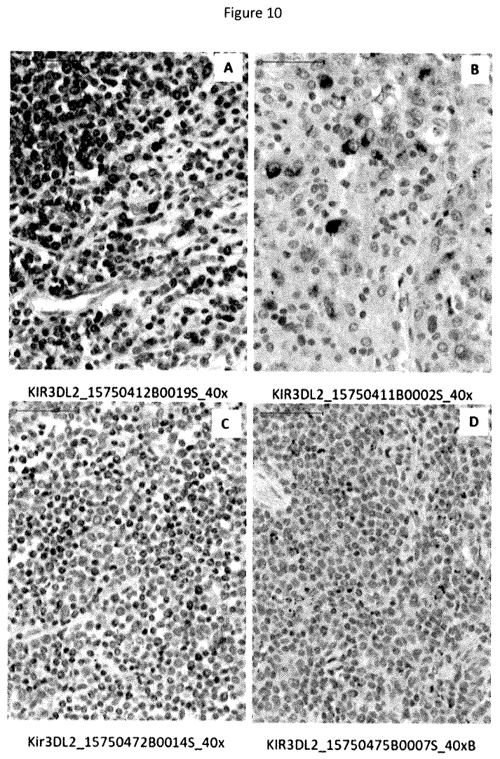

Additionally, further IHC stainings of PTCL tumor samples with the anti-KIR3DL2 clone P3-R4D-H5 according to the method presented above are shown in FIGS. 10A, 10B, 10C and 10D. FIG. 10A exhibits a highly KIR3DL2-positive PTCL-NOS tumor samples, whereas FIGS. 10C and 10D exhibit PTCL-NOS tumor samples with a moderate KIR3DL2 positivity (with a regional variability within the sample in FIG. 10D. FIG. 10B shows a PTCL-NOS tumor sample with scattered KIR3DL2-positive tumor cells against a backdrop of faint stromal cell KIR3DL2 positivity.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). Where “about” is used in connection with a number, this can be specified as including values corresponding to +/−10% of the specified number.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. An antibody or antibody fragment thereof capable of specifically binding to a KIR3DL2 polypeptide in a biological sample, wherein the antibody or antibody fragment comprises a heavy chain variable region comprising an amino acid sequence at least 80% identical to the amino acid sequence of SEQ ID NO: 21, and a light chain variable region comprising an amino acid sequence at least 80% identical to the amino acid sequence of SEQ ID NO: 22.

2-44. (canceled)


SEQUENCE LISTING (31 sequences)

SEQ ID NO: 1 - protein, 434 aa, Homo sapiens
LMGGQDKPFLSARPSTVVPRGGHVALQCHYRRGFNNFMLYKEDRSHVPIFHGRIFQESFIMGPVTPAHAGTYRCRGSRPHSLTGWSAPSNPLVIMVTGNHRKPSLLAHPGPLLKSGETVILQCWSDVMFEHFFLHRDGISEDPSRLVGQIHDGVSKANFSIGPLMPVLAGTYRCYGSVPHSPYQLSAPSDPLDIVITGLYEKPSLSAQPGPTVQAGENVTLSCSSWSSYDIYHLSREGEAHERRLRAVPKVNRTFQADFPLGPATHGGTYRCFGSFRALPCVWSNSSDPLLVSVTGNPSSSWPSPTEPSSKSGICRHLHVLIGTSVVIFLFILLLFFLLYRWCSNKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 2 - protein, 455 aa, Homo sapiens [sequence omitted]

SEQ ID NO: 3 - protein, 5 aa, Oryctolagus cuniculus
TYAMS

SEQ ID NO: 4 - protein, 7 aa, Oryctolagus cuniculus
GFSLSTY

SEQ ID NO: 5 - protein, 8 aa, Oryctolagus cuniculus
GFSLSTYA

SEQ ID NO: 6 - protein, 16 aa, Oryctolagus cuniculus
IIGASGNTWYASWAKG

SEQ ID NO: 7 - protein, 3 aa, Oryctolagus cuniculus
ASG

SEQ ID NO: 8 - protein, 7 aa, Oryctolagus cuniculus
IGASGNT

SEQ ID NO: 9 - protein, 18 aa, Oryctolagus cuniculus
FWAGYPSNAAATVSGMDP

SEQ ID NO: 10 - protein, 16 aa, Oryctolagus cuniculus
WAGYPSNAAATVSGMD

SEQ ID NO: 11 - protein, 20 aa, Oryctolagus cuniculus
ARFWAGYPSNAAATVSGMDP

SEQ ID NO: 12 - protein, 14 aa, Oryctolagus cuniculus
TLSTGYSVGSYGIG

SEQ ID NO: 13 - protein, 11 aa, Oryctolagus cuniculus
LSTGYSVGSYG

SEQ ID NO: 14 - protein, 9 aa, Oryctolagus cuniculus
TGYSVGSYG

SEQ ID NO: 15 - protein, 11 aa, Oryctolagus cuniculus
YHTEEIKHQGS

SEQ ID NO: 16 - protein, 7 aa, Oryctolagus cuniculus
YHTEEIK

SEQ ID NO: 17 - protein, 7 aa, Oryctolagus cuniculus
YHTEEIK

SEQ ID NO: 18 - protein, 13 aa, Oryctolagus cuniculus
ATAHGSGSSFHVV

SEQ ID NO: 19 - protein, 10 aa, Oryctolagus cuniculus
AHGSGSSFHV

SEQ ID NO: 20 - protein, 13 aa, Oryctolagus cuniculus
ATAHGSGSSFHVV

SEQ ID NO: 21 - protein, 123 aa, Oryctolagus cuniculus
QSLEESGGRLVTPGTPLTLTCTVSGFSLSTYAMSWVRQAPGKGLEWIGIIGASGNTWYASWAKGRFTISKTSTTVGLKITSPTTEDTATYFCARFWAGYPSNAAATVSGMDPWGPGTLVTVSS

SEQ ID NO: 22 - protein, 119 aa, Oryctolagus cuniculus
ELVLTQSPSLSASLDTTARLACTLSTGYSVGSYGIGWYQQVPGRPPRYLLTYHTEEIKHQGSGVPTRFSGSKDTSENTAVLSISGLQPEDEADYYCATAHGSGSSFHVVFGGGTQLTVT

SEQ ID NO: 23 - protein, 20 aa, Artificial Sequence
  Peptide designed in silico
CEHFFLHREGISEDPSRLVG

SEQ ID NO: 24 - protein, 20 aa, Artificial Sequence
  Peptide designed in silico
CTPLTDTSVYTELPNAEPRS

SEQ ID NO: 25 - protein, 13 aa, Artificial Sequence
  Peptide designed in silico
CPRAPQSGLEGVF

SEQ ID NO: 26 - protein, 118 aa, Artificial Sequence
  chimeric
QIQLVQSGSELKKPGASVKVSCKASGYTFTTAGMQWVRQAPGQGLEWIGWINSHSGVPKYAEDFKGRFVFSLDTSVSTAYLQISSLKAEDTAVYFCARGGDEGVMDYWGQGTTVTVSS

SEQ ID NO: 27 - protein, 107 aa, Artificial Sequence
  chimeric
DIQMTQSPSFLSASVGDRVTITCKASQDVSTAVAWYQQKPGQPPKLLIYWTSTRHTGVPDRFSGSGSGTDYTLTISSLQAEDVAVYYCQQHYSTPWTFGGGTKVEIK

SEQ ID NO: 28 - protein, 448 aa, Artificial Sequence
  chimeric
QIQLVQSGSELKKPGASVKVSCKASGYTFTTAGMQWVRQAPGQGLEWIGWINSHSGVPKYAEDFKGRFVFSLDTSVSTAYLQISSLKAEDTAVYFCARGGDEGVMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 214 aa, Artificial Sequence
  chimeric
DIQMTQSPSFLSASVGDRVTITCKASQDVSTAVAWYQQKPGQPPKLLIYWTSTRHTGVPDRFSGSGSGTDYTLTISSLQAEDVAVYYCQQHYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 444 aa, Homo sapiens
MSLMVVSMACVGLFLVQRAGPHMGGQDKPFLSAWPSAVVPRGGHVTLRCHYRHRFNNFMLYKEDRIHIPIFHGRIFQESFNMSPVTTAHAGNYTCRGSHPHSPTGWSAPSNPVVIMVTGNHRKPSLLAHPGPLVKSGERVILQCWSDIMFEHFFLHKEGISKDPSRLVGQIHDGVSKANFSIGPMMLALAGTYRCYGSVTHTPYQLSAPSDPLDIVVTGPYEKPSLSAQPGPKVQAGESVTLSCSSRSSYDMYHLSREGGAHERRLPAVRKVNRTFQADFPLGPATHGGTYRCFGSFRHSPYEWSDPSDPLLVSVTGNPSSSWPSPTEPSSKSGNPRHLHILIGTSVVIILFILLLFFLLHLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 31 - protein, 19 aa, Homo sapiens
TPLTDTSVYTELPNAEPRS